SEQUENCE LISTING (43 sequences)

SEQ ID NO: 1 - DNA, 356 bp, Artificial Sequence
  fragment of vector pGEP598 (Figure 1A)
tgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgagacggagctcagtctgaccgcggcgtctctggccggcatggtcccagcctcctcgctggcgccggctgggcaacatgcttcggcatggcgaatgggaccgatcgttcaaacatttggcaataaagtttcttaagattgaatcctgttgccggtcttgcgatgat

SEQ ID NO: 2 - DNA, 356 bp, Artificial Sequence
  fragment of vector pGEP560 (Figure 1B)
tgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtcatgtcttcatatgaagacattaatttctactaagtgtagatgagacggagctcagtctgaccgcggcgtctctggccggcatggtcccagcctcctcgctggcgccggctgggcaacatgcttcggcatggcgaatgggaccgatcgttcaaacatttggcaataaagtttcttaagattgaatcct

SEQ ID NO: 3 - DNA, 60 bp, Artificial Sequence
  oligonucleotide GEP1806
gatccaaattactgatgagtccgtgaggacgaaacgagtaagctcgtcatgtcttcatat

SEQ ID NO: 4 - DNA, 40 bp, Artificial Sequence
  Oligonucleotide GEP1807
gaagacattaatttctactaagtgtagatgagacggagct

SEQ ID NO: 5 - DNA, 32 bp, Artificial Sequence
  Oligonucleotide GEP1808
cgtttcgtcctcacggactcatcagtaatttg

SEQ ID NO: 6 - DNA, 60 bp, Artificial Sequence
  Oligonucleotide GEP1809
ccgtctcatctacacttagtagaaattaatgtcttcatatgaagacatgacgagcttact

SEQ ID NO: 7 - DNA, 53 bp, Artificial Sequence
  Oligonucleotide GEP1842
cgtcgtgctcccagttaccgtttttcagatgaaaacggtaactgggagcactt

SEQ ID NO: 8 - DNA, 53 bp, Artificial Sequence
  Oligonucleotide GEP1843
attaaagtgctcccagttaccgttttcatctgaaaaacggtaactgggagcac

SEQ ID NO: 9 - DNA, 53 bp, Artificial Sequence
  Oligonucleotide GEP1844
cgtcgtcttgcagtttctgcgatttcagatgaatcgcagaaactgcaagactt

SEQ ID NO: 10 - DNA, 53 bp, Artificial Sequence
  Oligonucleotide GEP1845
attaaagtcttgcagtttctgcgattcatctgaaatcgcagaaactgcaagac

SEQ ID NO: 11 - DNA, 57 bp, Artificial Sequence
  Oligonucleotide GEP1846
cgtccgaacatttacgagagattgattcagatgatcaatctctcgtaaatgttcgtt

SEQ ID NO: 12 - DNA, 57 bp, Artificial Sequence
  Oligonucleotide GEP 1847
attaaacgaacatttacgagagattgatcatctgaatcaatctctcgtaaatgttcg

SEQ ID NO: 13 - DNA, 29 bp, Artificial Sequence
  crGEP5.Sense
tagatctcgtcacgattcccctctcctgg

SEQ ID NO: 14 - DNA, 29 bp, Artificial Sequence
  crGEP5.Antisense
ggccccaggagaggggaatcgtgacgaga

SEQ ID NO: 15 - DNA, 298 bp, Artificial Sequence
  fragment of vector pGEP832 (Figure 4A)
tcgatgctcaccctgttgtttggtgttacttctgcagggatccaaattactgatgagtccgtgaggacgaaacgagtaagctcgtcgtcaaaagacctttttaatttctactcttgtagatgagacggagctcagtctgaccgcggcgtctctggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataat

SEQ ID NO: 16 - DNA, 298 bp, Artificial Sequence
  fragment of vector pGEP1062 (Figure 4B)
tcgatgctcaccctgttgtttggtgttacttctgcagggatccaaattactgatgagtccgtgaggacgaaacgagtaagctcgtcagagacgaaatatcgtctctgtcaaaagacctttttaatttctactcttgtagatgagacggagctcagtctgaccgcggcgtctctggccggcatggtcccagcctcctcgctggcgccggctgggcaacatgcttcggcatggcgaatgggaccgatcgttcaaacatttggcaataaagtttcttaagattgaatcctgttgccggtct

SEQ ID NO: 17 - DNA, 60 bp, Artificial Sequence
  Oligonucleotide GEP1901
gatccaaattactgatgagtccgtgaggacgaaacgagtaagctcgtcagagacgaaata

SEQ ID NO: 18 - DNA, 54 bp, Artificial Sequence
  Oligonucleotide GEP1902
tcgtctctgtcaaaagacctttttaatttctactcttgtagatgagacggagct

SEQ ID NO: 19 - DNA, 46 bp, Artificial Sequence
  Oligonucleotide GEP1903
ctgacgagcttactcgtttcgtcctcacggactcatcagtaatttg

SEQ ID NO: 20 - DNA, 60 bp, Artificial Sequence
  Oligonucleotide GEP1904
ccgtctcatctacaagagtagaaattaaaaaggtcttttgacagagacgatatttcgtct

SEQ ID NO: 21 - DNA, 57 bp, Artificial Sequence
  Oligonucleotide GEP1890
cgtcgaactcgctgatgggatatctttcagatgaagatatcccatcagcgagttctt

SEQ ID NO: 22 - DNA, 57 bp, Artificial Sequence
  Oligonucleotide GEP1911
tgacaagaactcgctgatgggatatcttcatctgaaagatatcccatcagcgagttc

SEQ ID NO: 23 - DNA, 57 bp, Artificial Sequence
  Oligonucleotide GEP1892
cgtcgatatcaagttggcttcctccttcagatgaggaggaagccaacttgatatctt

SEQ ID NO: 24 - DNA, 57 bp, Artificial Sequence
  Oligonucleotide GEP1912
tgacaagatatcaagttggcttcctcctcatctgaaggaggaagccaacttgatatc

SEQ ID NO: 25 - DNA, 57 bp, Artificial Sequence
  Oligonucleotide GEP1894
cgtccttggtgtaattgtcatagatttcagatgaatctatgacaattacaccaagtt

SEQ ID NO: 26 - DNA, 57 bp, Artificial Sequence
  Oligonucleotide GEP1913
tgacaacttggtgtaattgtcatagattcatctgaaatctatgacaattacaccaag

SEQ ID NO: 27 - DNA, 26 bp, Artificial Sequence
  m7GEP1.Sense
tagatctcgtcacgattcccctctcc

SEQ ID NO: 28 - DNA, 26 bp, Artificial Sequence
  m7GEP1.Antisense
ggccggagaggggaatcgtgacgaga

SEQ ID NO: 29 - DNA, 35 bp, Artificial Sequence
  MAD7 Scaffold  sequence
gtcaaaagacctttttaatttctactcttgtagat

SEQ ID NO: 30 - DNA, 19 bp, Artificial Sequence
  mature MAD7 Scaffold sequence
aatttctactcttgtagat

SEQ ID NO: 31 - DNA, 37 bp, Artificial Sequence
  HH ribozyme encoding sequence
ctgatgagtccgtgaggacgaaacgagtaagctcgtc

SEQ ID NO: 32 - RNA, 37 nt, Artificial Sequence
  HH ribozyme sequence
cugaugaguccgugaggacgaaacgaguaagcucguc

SEQ ID NO: 33 - DNA, 53 bp, Oryza sativa
ccgccaacactgccaatgccggtcccaagcccggataaaagtggagggggcgg

SEQ ID NO: 34 - RNA, 53 nt, Oryza sativa
ccgccaacacugccaaugccggucccaagcccggauaaaaguggagggggcgg

SEQ ID NO: 35 - DNA, 63 bp, Helianthus annuus
gcggggggcgtcagtcctactctgcacctcctcgtggtgtcgcctgggaaccctctttcgcaa

SEQ ID NO: 36 - RNA, 63 nt, Helianthus annuus
gcggggggcgucaguccuacucugcaccuccucguggugucgccugggaacccucuuucgcaa

SEQ ID NO: 37 - DNA, 86 bp, Helianthus annuus
gcggggggcgtcagtcctactctgcacctcctcgtggtgtcgcctgggaaccctctttcgcaagaaagaggagccaagcagagagg

SEQ ID NO: 38 - RNA, 86 nt, Helianthus annuus
gcggggggcgucaguccuacucugcaccuccucguggugucgccugggaacccucuuucgcaagaaagaggagccaagcagagagg

SEQ ID NO: 39 - DNA, 80 bp, Cynara scolymus
ggcgtcagtcctactctgcacctcctcgtggtgtcgcctgggaaccctctttcacaagaaagaggagccaagcagagagg

SEQ ID NO: 40 - RNA, 80 nt, Cynara scolymus
ggcgucaguccuacucugcaccuccucguggugucgccugggaacccucuuucacaagaaagaggagccaagcagagagg

SEQ ID NO: 41 - DNA, 68 bp, Hepatitis D virus
ggccggcatggtcccagcctcctcgctggcgccggctgggcaacatgcttcggcatggcgaatgggac

SEQ ID NO: 42 - RNA, 68 nt, Hepatitis D virus
ggccggcauggucccagccuccucgcuggcgccggcugggcaacaugcuucggcauggcgaaugggac

SEQ ID NO: 43 - DNA, 21 bp, Artificial Sequence
  LbCpf1 crRNA repeat
taatttctactaagtgtagat

(19) United States
(12) Patent Application Publication
(10) Pub. No.: US 2021/0139926 A1
(43) Pub. Date: May 13, 2021

(54) PLANT GENOME EDITING

(71) Applicant: KWS SAAT SE & CO. KGaA, Einbeck (DE)

(72) Inventor: Brent TRAUTERMAN, Saint Louis, MO (US)

(73) Assignee: KWS SAAT SE & CO. KGaA, Einbeck (DE)

(21) Appl. No.: 17/005,090

(22) Filed: Aug. 27, 2020

Related U.S. Application Data
(60) Provisional application No. 62/893,518, filed on Aug. 29, 2019.

(51) Int. Cl. C12N 15/82; C12N 15/11; C12N 15/113

(52) U.S. Cl. CPC C12N 15/8218; C12N 15/11; C12N 2310/121; C12N 2310/20; C12N 2310/14; C12N 15/113

(57) ABSTRACT

The present invention relates to a nucleic acid cassette for improved genome editing by transiently silencing non-homologous end joining (NHEJ) and/or microhomology-mediated end joining (MMEJ) pathways during the editing process. A nucleic acid cassette according to the invention comprises (a) at least one RNA interference (RNAi) component, (b) at least one CRISPR guide RNA component and (c) at least one RNA activating unit, wherein the nucleic acid cassette is suitable for the simultaneous expression of at least one sequence encoding an RNA interference (RNAi) component and at least one sequence encoding a CRISPR guide RNA component. The invention further provides methods for modification of at least one genomic target sequence in a cell, wherein a nucleic acid cassette according to the invention and an RNA-guided site-specific nuclease is introduced in the cell and editing of the target sequence takes place while NHEJ and/or MMEJ pathways are silenced.

### CROSS-REFERENCE TO RELATED APPLICATIONS

This application is a non-provisional of Provisional Patent Application No. 62/893,518, filed on Aug. 29, 2019. The entire contents of this application are incorporated herein by reference in its entirety.

### SEQUENCE LISTING

This application contains a Sequence Listing which has been submitted in ASCII format via EFS-Web and is hereby incorporated by reference herein in its entirety. The ASCII text file was created on Aug. 29, 2019, is named SQL_KWS0341US_ST25.txt and is 10,216 bytes in size.

### TECHNICAL FIELD

The present invention relates to a nucleic acid cassette for improved genome editing by transiently silencing non-homologous end joining (NHEJ) and/or microhomology-mediated end joining (MMEJ) pathways during the editing process. A nucleic acid cassette according to the invention comprises (a) at least one RNA interference (RNAi) component, (b) at least one CRISPR guide RNA component and (c) at least one RNA activating unit, wherein the nucleic acid cassette is suitable for the simultaneous expression of at least one sequence encoding an RNA interference (RNAi) component and at least one sequence encoding a CRISPR guide RNA component. The invention further provides methods for modification of at least one genomic target sequence in a cell, wherein a nucleic acid cassette according to the invention and an RNA-guided site-specific nuclease is introduced in the cell and editing of the target sequence takes place while NHEJ and/or MMEJ pathways are silenced. Further provided are cells and organisms obtainable by such a method as well as a method of producing a nucleic acid cassette according to the invention.

### BACKGROUND OF THE INVENTION

In the past, plant breeding has been a rather cumbersome procedure, whereby new varieties are produced from a cross between parental plants or through self-pollination. The process is based on identifying a desired characteristic in one plant or germplasm. Particular traits of interest are, for example, higher resistance to biotic and abiotic stress, e.g., drought, plant diseases etc., and higher yields and vigor. Crossings with another plant then allows the desired trait to appear in the offspring. Necessarily, unwanted characteristics are co-transferred to the offspring as well, which requires several more breeding cycles in order to be replaced by desired traits. The traditional form of breeding, even though more and more assisted by molecular techniques (e.g., marker-assisted breeding) is rather time consuming to accomplish. Still, due to climate change and an increasing demand for food security, safe, precise and efficient techniques to speed up plant breeding processes are urgently needed.

Over the last decades, the use of highly specific site-directed nucleases (SDNs) or modified variants thereof became more and more common in modern plant breeding during so-called genome editing or engineering (GE) to introduce double-strand breaks (DSBs), single-strand nicks, or targeted base pair exchanges and to optionally repair the lesion in a targeted manner. SDNs used in plant biotechnology are, for instance, meganucleases, Zinc-Finger Nucleases (ZFNs), Transcription Activator Like Effector Nucleases (TALENs), and Clustered Regularly Interspaced Short Palindromic Repeats (CRISPR) nuclease-based systems. Depending on the outcome of GE, the corresponding procedure is classified into several groups. Whereas SDN-1 is understood to produce a double-stranded break in the genome of a plant without the addition of foreign DNA, SDN-2 produces a DSB, and while the break is repaired by the cell, a small nucleotide template (repair template(RT)) is supplied that is complementary to the area of the break, which in turn, is used by the cell to repair the break. Finally, SDN-3 also induces a DSB in the DNA, but is accompanied by a template containing a gene or other sequence of genetic material. The cell's natural repair process then utilizes this template to repair the break; resulting in the introduction of the genetic material.

Still, for all GE approaches, the major unknown and hard to control variable are endogenous repair mechanisms in a eukaryotic cell, in particular a plant cell. A further challenge is the propensity for introduced RTs to integrate randomly into the genome at unpredictable and uncontrollable locations. Therefore, more precise, controllable and predictable techniques for plant GE are urgently needed.

Non-homologous end joining (NHEJ) is the major pathway for repair of DNA double-strand breaks (DSBs) in eukaryotic cells, including human and plant cells. NHEJ does not require homologous sequences but is error-prone and often leads to insertions or deletions thus hampering targeted GE approaches. A variety of different enzymes have been identified which are involved in NHEJ in eukaryotic cells, including Ku70, Ku80, DNA-dependent protein kinase, Ataxia telangiectasia mutated (ATM), ATM—and Rad3—related (ATR), Artemis, XRCC4, DNA ligase IV and XLF and PAXX.

The Ku70/Ku80 heterodimer represents the core of the NHEJ machinery, which binds to DNA double-strand break ends and interacts with other NHEJ proteins such as DNA ligase IV and XRCC4-like factor (XLF), which are in involved in double-strand break repair. DNA dependent protein kinase, catalytic subunit (DNA-PKcs) relies on Ku70/Ku80 to direct it to DNA ends and induce its kinase activity, which is required for NHEJ. It is furthermore suggested that PAXX assists to promote and stabilize the assembly of the NHEJ complex (Liu et al., PAXX promotes KU accumulation at DNA breaks and is essential for end-joining in XLF-deficient mice; Nature Communications 8, Article Number: 13816 (2017)).

Ataxia telangiectasia and Rad3-related protein (ATR) is a phosphatidylinositol 3-kinase-related kinase, which is activated in response to DNA breaks. Ataxia telangiectasia-mutated (ATM) kinase and Artemis, a protein encoded by DNA cross link repair 1C (DCLRE1C) gene, are both required for repair of certain double-strand breaks.

A further element involved in DNA repair is Polymerase θ (Polymerase theta, Pol θ, or Pol theta), encoded by the POLQ gene (e.g., for plants see: van Kregten et al., 2016, T-DNA integration in plants results from polymerase-θ-mediated DNA repair. Nature Plants 2, Article number: 16164). Polymerase θ was formerly considered a NHEJ component but has recently been implicated in microhomology-mediated end joining (MMEJ) processes, which are associated with insertions at the beak side (Sfeir and Symington, Trends in Biochemical Sciences, 40 (11), 701-714 (2015)).

Polymerase θ in mammals is an atypical A-family type polymerase with an N-terminal helicase-like domain, a large central domain harboring a Rad51 interaction motif, and a C-terminal polymerase domain capable of extending DNA strands from mismatched or even unmatched termini. DNA molecules can be randomly incorporated into eukaryotic genomes through the action of Pol θ being a low fidelity polymerase (Hogg et al., 2012. Promiscuous DNA synthesis by human DNA polymerase θ. Nucleic Acids Research, Volume 40, Issue 6, 1 Mar. 2012, Pages 2611-2622) that is required for random integration of T-DNAs in plants. Knockout mutant plants lacking Pol θ activity are incapable of integrating T-DNA molecules during Agrobacterium tumefaciens mediated plant transformation (van Kregten et al., 2016, supra). In vitro experiments identified an evolutionarily conserved loop in the polymerase domain that is essential for synapsing DNA ends during end joining to protect the genome against gross chromosomal rearrangements (Sfeir, The FASEB Journal, vol. 30, no. 1, 2016).

Microhomology-mediated end joining (MMEJ) or alternative non-homologous end joining (ANHEJ) relies on microhomologous sequences for alignment of the broken ends before joining. The broken ends are resected by MRE nuclease resulting in single-stranded overhangs, which anneal at short complementary regions of only a few nucleotides (microhomologies). For example, DSBs in plant organelles are repaired via genomic rearrangements characterized by microhomologous repeats (Garcia-Medel et al., Nucleic Acids Research, vol. 47(6), 8 Apr. 2019, 3028-3044). The authors show that organellar DNA polymerases from Arabidopsis thaliana (AtPollA and AtPollB) perform MMEJ using microhomologous sequences as short as six nucleotides. After annealing, overhanging bases are removed and the gaps are filled in by Polymerase θ. MMEJ is highly mutagenic and it often results in deletions, insertions and translocations (Sfeir and Symington (2015), supra).

Components involved in MMEJ pathways include—besides Polymerase θ—organellar family-A DNA polymerases (DNAPs), which have been implicated in strand displacement, stabilizing the pairing of microhomologous regions (Baruch-Torres and Brieba, Nucleic Acid Research, 2017, 45 (18), 10751-10763), DNA ligase III, which ligates the break ends after the gaps have been filled, and Poly-ADP ribose polymerase (PARP1), which binds to broken DNA ends and is able to tether DNA fragments (Sfeir and Symington (2015), supra).

Since GE strategies rely on site-directed nucleases (SDNs) to introduce a double-strand break at a specific target site to be modified, NHEJ and MMEJ pathways strongly interfere with the editing as they tend to repair the double-strand break in a highly unpredictable manner. It would therefore be advantageous to tip the DNA repair pathway towards a desired outcome, which, in most cases, is a homology-directed repair with a predictable result. On the other hand, non-homologous DNA repair pathways are essential for an organism's survival. An irreversible shutdown of NHEJ and MMEJ pathways is therefore also not desirable.

It was an aim of the present invention to increase the predictability and precision of GE approaches by providing means and methods to suppress NHEJ and MMEJ pathways during the editing process. The suppression should preferably be transient so that the resulting organism is still able to perform essential DNA repair. Moreover, it was an aim of the present invention that the GE components and the components, which act to suppress NHEJ and MMEJ can be delivered to a target cell and expressed therein in a simple and concerted way.

### SUMMARY OF THE INVENTION

In a first aspect the present invention provides a nucleic acid cassette, wherein the nucleic acid cassette comprises
(a) at least one RNA interference (RNAi) component;
(b) at least one CRISPR guide RNA component; and
(c) at least one RNA activating unit;
   wherein the nucleic acid cassette is suitable for the simultaneous expression of at least one sequence encoding an RNA interference (RNAi) component and at least one sequence encoding a CRISPR guide RNA component.

In one embodiment according to the various aspects of the present invention, the RNAi interference component encodes at least one short-hairpin (shRNA) sequence, wherein the at least one shRNA sequence targets at least one non-homologous end joining (NHEJ) pathway component and/or at least one microhomology-mediated end joining (MMEJ) component.

In another embodiment according to the various aspects of the present invention, the at least one NHEJ pathway component encodes a sequence selected from the group consisting of Ku70, Ku80, DNA-dependent protein kinase, Ataxia telangiectasia mutated (ATM), ATM—and Rad3—related (ATR), Artemis, XRCC4, DNA ligase IV and XLF, PAXX, or any combination thereof.

In a further embodiment according to the various aspects of the present invention, the at least one MMEJ pathway component encodes a sequence selected from the group consisting of Polymerase theta (pol Θ), an organellar family-A DNA polymerase, DNA ligase III, PARP-1, or any combination thereof.

In yet another embodiment according to the various aspects of the present invention, the RNAi interference component encodes more than one short-hairpin (shRNA) targeting more than one NHEJ pathway component(s) and/or MMEJ pathway component(s).

In one embodiment according to the various aspects of the present invention, the RNAi interference component is encoded by a sequence comprising at least one nucleic acid sequence of any one of SEQ ID NOs: 7 to 12, or SEQ ID NOs: 21 to 26 or a nucleic acid sequence having at least 90%, 91%, 92%, 93%, 94%, 95%, 96%, 97%, 98% or 99% sequence identity to any one of SEQ ID NOs: 7 to 12, or SEQ ID NOs: 21 to 26.

In another embodiment of the various aspects of the present invention, the CRISPR guide RNA component comprises a sequence encoding a scaffold region and a targeting region.

In a further embodiment of the various aspects of the present invention, the CRISPR guide RNA component comprises at least one scaffold region encoded by a sequence selected from a nucleic acid sequence of any one of SEQ ID NOs: 29, 30 or 43 or a nucleic acid sequence having at least 90%, 91%, 92%, 93%, 94%, 95%, 96%, 97%, 98% or 99% sequence identity to any one of SEQ ID NOs: 29, 30 or 43.

In yet another embodiment according to the various aspects of the present invention, the CRISPR guide RNA component comprises at least one targeting region encoded by a sequence selected from a nucleic acid sequence of any one of SEQ ID NOs: 13, 14, 27, or 28 or a nucleic acid sequence having at least 90%, 91%, 92%, 93%, 94%, 95%, 96%, 97%, 98% or 99% sequence identity to any one of SEQ ID NOs: 13, 14, 27, or 28.

In one embodiment according to the various aspects of the present invention, the at least one RNA activating unit comprises at least one ribozyme system, wherein the at least one ribozyme system comprises at least one sequence encoding a self-cleaving ribozyme.

In another embodiment according to the various aspects of the present invention, the at least one RNA activating unit comprises at least one ribozyme system comprising at least a pair of sequences encoding two self-cleaving ribozymes, or wherein the ribozyme system comprises at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or more, sequences encoding a self-cleaving ribozyme.

In a further embodiment according to the various aspects of the present invention, the at least one self-cleaving ribozyme of the ribozyme system, or the sequence encoding the same, is independently selected from the group consisting of a hammerhead ribozyme (SEQ ID NOs: 31 and 32), a hairpin ribozyme, a hepatitis-delta virus (HDV) ribozyme (SEQ ID NOs: 41 and 42), a sunflower ribozyme (SEQ ID NOs: 35 to 38), an artichoke ribozyme (SEQ ID NOs: 39 and 40), a rice ribozyme (SEQ ID NOs: 33 and 34), a VS ribozyme, a self-splicing group I intron, or RNase P, a ribozyme sequence of Tobacco ringspot virus (TRSV) satellite RNA, or a combination thereof.

In one embodiment according to the various aspects of the present invention, the at least one RNA activating unit comprises at least one ribonuclease recognition site, wherein the at least one ribonuclease recognition site is recognized and cleaved by at least one RNA-guided site-specific nuclease (SSN) guided by the at least one CRISPR guide RNA component.

In another embodiment according to the various aspects of the present invention, the nucleic acid cassette comprises more than one RNA interference (RNAi) component and/or more than one CRISPR guide RNA component, wherein the sequence encoding an RNAi component and encoding a CRISPR guide RNA component are
(i) each flanked by a sequence encoding a self-cleaving ribozyme at the 5′ and at the 3′ end, or wherein
(ii) a sequence encoding an RNAi component and encoding a CRISPR guide RNA component is flanked by a sequence encoding a self-cleaving ribozyme at the 5′ and at the 3′ end;
   wherein the self-cleaving ribozyme is independently selected from the group consisting of a hammerhead ribozyme (SEQ ID NOs: 31 and 32), a hairpin ribozyme, a hepatitis-delta virus (HDV) ribozyme (SEQ ID NOs: 41 and 42), a sunflower ribozyme (SEQ ID NOs: 35 to 38), an artichoke ribozyme (SEQ ID NOs: 39 and 40), a rice ribozyme (SEQ ID NOs: 33 and 34), a VS ribozyme, a self-splicing group I intron, RNase P, or a ribozyme sequence of Tobacco ringspot virus (TRSV) satellite RNA.

In yet another embodiment according to the various aspects of the present invention, the nucleic acid cassette comprises a single promoter driving the expression of the at least one RNA interference (RNAi) component, the at least one CRISPR guide RNA component, and the at least one RNA activating unit.

In one embodiment according to the various aspects of the present invention, the nucleic acid cassette comprises a promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof.

In another embodiment according to the various aspects of the present invention, the nucleic acid cassette comprises at least one intron.

In one embodiment according to the various aspects of the present invention, the at least one intron is selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

In a further embodiment according to the various aspects of the present invention, the nucleic acid cassette comprises a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and a AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

In another embodiment according to the various aspects of the present invention, the nucleic acid cassette comprises at least one terminator selected from the group consisting of nosT, a double 35S terminator, a ZmEf1 terminator, an AtSac66 terminator, an octopine synthase (ocs) terminator, or a pAG7 terminator, or a combination thereof.

According to another aspect the present invention relates to a vector encoding the nucleic acid cassette according to any of the embodiments described above.

In a further aspect, the present invention relates to a method for the targeted modification of at least one genomic target sequence in a cell, wherein the method comprises the following steps:
(a) introducing into the cell at least one nucleic acid cassette as defined in any of the embodiments above under conditions allowing the expression of the at least one RNA interference (RNAi) component, the at least one CRISPR guide RNA component, and the at least one RNA activating unit, and thus under conditions allowing activation of the at least one RNA activating unit;
(b) transiently silencing at least one non-homologous end joining (NHEJ) pathway component and/or at least one microhomology-mediated end joining (MMEJ) component through the action of at least one short-hairpin (shRNA) sequence encoded by the at least one RNA interference (RNAi) component;
(c) introducing into the cell at least one site-specific nuclease (SSN), or a sequence encoding the same, wherein the site-specific nuclease has the capability of inducing a single- or double-strand break at a genomic target sequence upon interaction with a sequence encoded by the at least one CRISPR guide RNA component;
(d) optionally: determining the expression level of the at least one NHEJ pathway component and/or at least one MMEJ component, and/or determining the presence of the at least one targeted modification of at least one genomic target sequence in the cell;
(e) obtaining a cell comprising a targeted modification of at least one genomic target sequence.

In one embodiment, the method additionally comprises the introduction of at least one single- or double-stranded repair template (RT), or a sequence encoding the same, simultaneously with, or before, or after step (a).

In one embodiment of the method described above, the at least one NHEJ pathway component encodes a sequence selected from the group consisting of Ku70, Ku80, DNA-dependent protein kinase, Ataxia telangiectasia mutated (ATM), ATM—and Rad3—related (ATR), Artemis, XRCC4, DNA ligase IV and XLF, PAXX, or any combination thereof.

In another embodiment of the method described above, the at least one MMEJ pathway component encodes a sequence selected from the group consisting of Polymerase theta (pol Θ), an organellar family-A DNA polymerase, DNA ligase III, PARP-1, or any combination thereof.

In a further embodiment of the method described above, the at least one site-specific nuclease (SSN) is an RNA-guided nuclease.

In one embodiment of the method described above, the at least one site-specific nuclease (SSN) is selected from a nuclease from a CRISPR/Cas system, preferably from a CRISPR/Cfp1 system, a CRISPR/MAD7 system, a CRISPR/MAD2 system, a CRISPR/Cas9 system, a CRISPR/CasX system, or a CRISPR/CasY system.

In another embodiment of the method described above, the at least one site-specific nuclease (SSN) is selected from the group consisting of a CRISPR/Cfp1 system from Lachnospiraceae bacterium ND2006 (LbCpf1), a CRISPR/Cfp1 system from a Acidaminococcus sp. BV3L6 (AsCpf1), or a CRISPR/MAD7 system from Eubacterium rectale.

In a further embodiment, the at least one site-specific nuclease (SSN), or the sequence encoding the same, is introduced simultaneously with, before or after step (a).

In one embodiment of the method described above, the at least one nucleic acid cassette and/or the at least one site-specific nuclease (SSN), or the sequence encoding the same, are stably or transiently introduced and expressed in the cell.

In another embodiment of the method described above, the targeted modification of the at least one genomic target sequence in a cell is selected from at least one point mutation, at least one insertion, or at least one deletion, or any combination thereof.

In a further embodiment of the method described above, the cell is a eukaryotic cell, preferably a plant cell, or an animal cell.

In one embodiment of the method described above, the cell is a plant cell originating from a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia foumieri, Spinacia oleracea, Allium cepa, Allium fistulosum, Allium sativum, and Allium tuberosum.

In another aspect, the present invention relates to a cell, preferably a eukaryotic cell selected from a plant cell or an animal cell, obtainable by a method according to any of the embodiments described above.

In yet another aspect, the present invention relates to an or organism, or part of an organism, obtainable by cultivating a cell as described above.

In yet a further aspect, the present invention relates to a method of producing a nucleic acid cassette according to any of the embodiments described above.

In one embodiment, the method of producing a nucleic acid cassette according to any of the embodiments described above comprises a step of inserting at least one unique cloning site into a nucleic acid vector to provide at least one flexible insertion site for at least one short-hairpin (shRNA) sequence of an RNA interference (RNAi) component and/or for at least one targeting region of a CRISPR guide RNA component.

### BRIEF DESCRIPTIONS OF THE DRAWINGS

FIG. 1A: Fragment of vector pGEP598 comprising expression cassette of 5′ Hammerhead ribozyme and LbCpf1 crRNA repeat (1A; SEQ ID NO: 1).

FIG. 1B: Fragment of vector pGEP960 containing additional BbsI sites (1B; SEQ ID NO: 2).

FIG. 2: Vector containing the ZmUbi1 promoter+intron driving expression of a Hammerhead Ribozyme+LbCpf1 repeat+HDV Ribozyme into a nos terminator and inserted siGEP3 and crGEP5 (pGEP987).

FIG. 3: Knockdown % of Ku80 RNA. Black columns represent raw data from qPCR results. White columns represent RNA knockdown normalized to transformation efficiency.

FIG. 4A: Fragment of vector pGEP832 comprising expression cassette of 5′ Hammerhead ribozyme and LbMAD7 Scaffold (4A; SEQ ID NO: 15)

FIG. 4B: and fragment of vector pGEP1062 containing additional Esp3I (BsmBI) sites (4B; SEQ ID NO: 16).

FIG. 5: Vector containing the ZmUbi1 promoter+intron driving expression of a Hammerhead Ribozyme+Mad7 guide repeat+HDV Ribozyme into a nos terminator and inserted siGEP6 and m7GEP1 (pGEP1072).

FIG. 6: Knockdown % of PolTheta RNA. Black columns represent raw data from qPCR results. White columns represent RNA knockdown normalized to transformation efficiency.

### DEFINITIONS

A “nucleic acid cassette” is a nucleic acid sequence that encodes two or more components such as genes, regulatory sequences or RNA based effectors such as gene silencing constructs or ribozymes. Furthermore, the “nucleic acid cassette” may comprise (a) recombination site(s) for integration into a vector or a genomic sequence. Preferably, a nucleic acid cassette comprises all necessary elements to express or transcribe the one or more components, such as at least one promoter and at least one terminator. A single promoter in the nucleic acid cassette may simultaneously drive transcription of preferably all the components encoded in the nucleic acid cassette.

A “simultaneous expression” of the components of the nucleic acid cassette means that the all the components are transcribed and are able to exert their function in a concerted manner. “Expression” in this context includes transcription as well as translation.

The terms “RNA interference” or “RNAi” as used herein interchangeably refer to a gene down-regulation (or knockdown) mechanism meanwhile demonstrated to exist in all eukaryotes. The mechanism was first recognized in plants where it was called “post-transcriptional gene silencing” or “PTGS”. In RNAi, small RNAs (of about 21-24 nucleotides) function to guide specific effector proteins (e.g., members of the Argonaute protein family) to a target nucleotide sequence by complementary base pairing. The effector protein complex then down-regulates the expression of the targeted RNA or DNA. Small RNA-directed gene regulation systems were independently discovered (and named) in plants, fungi, worms, flies, and mammalian cells. Collectively, PTGS, RNA silencing, and co-suppression (in plants); quelling (in fungi and algae); and RNAi (in Caenorhabditis elegans, Drosophila, and mammalian cells) are all examples of small RNA-based gene regulation systems.

An “RNA interference (RNAi) component” or “RNAi component” in the context of the present invention refers to an RNA molecule, which is capable to induce RNA interference, i.e. down-regulation of the expression of a target gene. Such RNA molecules may be microRNAs (miRNAs) or small interfering RNAs (siRNAs). Preferably, an RNA interference (RNAi) component is a short hairpin RNA (shRNA), which can silence a specific target gene via RNA interference. Therefore, an shRNA “targets” a gene when it down-regulates the expression (transcription or translation) of that specific gene.

The term “transiently silencing” as used herein means that the RNA interference (RNAi) component is expressed only temporarily and silences its target gene(s) preferably only during the editing process. Accordingly, the RNA interference (RNAi) component is preferably introduced into a cell under conditions so that preferably no integration into the endogenous nucleic acid material of the cell takes place and the RNAi component is not inherited to a progeny of the cell. The target genes are fully expressed again once the RNAi component is not present in the cell anymore.

In the context of the present invention, a “CRISPR guide RNA component” is an RNA molecule that recruits and guides a site-specific CRISPR nuclease in a sequence-dependent manner to a specific target site, where the CRISPR nuclease introduces a double-strand break. The CRISPR guide RNA component may comprise a trans-activating CRISPR RNA (tracrRNA) and a CRISPR RNA (crRNA) or it may be a single guide RNA (sgRNA) consisting of a crRNA and a tracrRNA in one construct. Certain CRISPR nucleases may only need a crRNA for target recognition and cleavage. Preferably, the CRISPR guide RNA component comprises a crRNA, which is sufficient by itself and does not require the presence of a tracrRNA for targeting. Furthermore, the crRNA may be processed by its associated site-specific nuclease without the need for further effectors (e.g. Cpf1 and MAD7).

A CRISPR guide RNA component comprises a “scaffold region” and a “target region”. The “scaffold region” is a sequence, to which the RNA-guided site-specific nuclease binds to form a targetable nuclease complex. The scaffold region may comprise direct repeats, which are recognized and processed by the site-specific nuclease to provide mature crRNA. Thus, the scaffold region may comprise at least one “ribonuclease recognition site”, which may also be part of the RNA activating unit of the nucleic acid cassette according to the invention. The “target region” defines the complementarity to the intended modification site for the genome editing and is provided in the nucleic acid cassette as a spacer with a specifically designed sequence.

An “RNA activating unit” in the context of the present invention represents an element within a nucleic acid cassette, which is suitable to induce processing of at least one RNAi component and/or at least one CRISPR guide RNA component so that the respective RNA components as effector molecules can exert their effects or silencing and/or CRISPR nuclease guidance. The RNA activating unit may comprise at least one ribozyme system comprising at least one self-cleaving ribozyme sequence, which upon transcription cleaves the transcript at a specific site thus e.g. releasing and thereby activating one or more of the component(s) encoded in the nucleic acid cassette. Alternatively, or in addition to the ribozyme system, the RNA activating unit may encode at least one “ribonuclease recognition site”, e.g. comprising direct repeats, which is recognized and cleaved by an RNA-guided site-specific nuclease. In this case the RNA-guided site-specific nuclease (e.g. Cpf1 or MAD7) is guided by the at least one CRISPR guide RNA component encoded in the nucleic acid cassette. Upon transcription of the ribonuclease recognition site and introduction of RNA-guided site-specific nuclease, the nuclease processes the transcript to activate its associated crRNA and/or to release and thus activate further components encoded in the nucleic acid cassette.

A self-cleaving ribozyme is an RNA molecule that is capable to catalyze its own cleavage at a specific site. Upon transcription, self-cleaving ribozymes fold into a specific structure, sometimes requiring the presence of certain metal cations, which induces cleavage of the phosphodiester backbone at a certain position. A number of ribozymes are known, which can be used in a variety of settings. A “self-cleaving ribozyme system” may comprise one or more self-cleaving ribozymes, which is/are arranged within a nucleic acid cassette so that upon transcription of the nucleic acid cassette, the RNA is cleaved in one or more specific positions. For example, a certain component may be released from the nucleic acid cassette and thus activated by the action of two flanking self-cleaving ribozymes forming a self-cleaving ribozyme system. A self-cleaving ribozyme system may also comprise three or more self-cleaving ribozymes that are arranged in the nucleic acid cassette so that they can activate the components.

Conditions “allowing activation” of the at least one RNA activating unit as used herein means that the RNA activating unit is transcribed and folds into a structure, which facilitates self-cleavage in case the RNA activating unit comprises at least one self-cleaving ribozyme. Such conditions may e.g. require the presence of certain metal cations as explained above. In case the RNA activating unit encodes at least one ribonuclease recognition site, the ribonuclease recognition site is transcribed and is accessible to be recognized and cleaved by a RNA-guided site-specific nuclease.

A “non-homologous end joining (NHEJ) pathway component” or “NHEJ component” or a “microhomology-mediated end joining (MMEJ) pathway component” or “MMEJ component” refers to an element, which has been shown to play a role in NHEJ or MMEJ so that reducing or inhibiting the expression of the element will reduce or abolish NEHJ or MMEJ events.

A “targeted modification” of at least one genomic target sequence in the context of the present invention refers to any change of a (nucleic acid) sequence that results in at least one difference in the (nucleic acid) sequence distinguishing it from the original sequence. In particular, a modification can be achieved by insertion or addition of one or more nucleotide(s), or substitution or deletion of one or more nucleotide(s) of the original sequence or any combination of these.

A “eukaryotic cell” as used herein refers to a cell having a true nucleus, a nuclear membrane and organelles belonging to any one of the kingdoms of Protista, Plantae, Fungi, or Animalia. Eukaryotic organisms can comprise monocellular and multicellular organisms. Preferred eukaryotic cells and organisms according to the present invention are animal cells and plant cells or animals and plants.

The terms “plant” or “plant cell” as used herein refer to a plant organism, a plant organ, differentiated and undifferentiated plant tissues, plant cells, seeds, and derivatives and progeny thereof. Plant cells include without limitation, for example, cells from seeds, from mature and immature embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. The different eukaryotic cells, for example, animal cells, fungal cells or plant cells, can have any degree of ploidity, i.e. they may either be haploid, diploid, tetraploid, hexaploid or polyploid.

A “site-specific nuclease” herein refers to a nuclease or an active fragment thereof, which is capable to specifically recognize and cleave DNA at a certain location. This location is herein also referred to as a “target sequence”. Such nucleases typically produce a double-strand break (DSB), which is then repaired by nonhomologous end-joining (NHEJ) or homologous recombination (HR). The nucleases CRISPR/Cas systems, CRISPR/Cpf1 systems, CRISPR/C2C2 systems, CRISPR/CasX systems, CRISPR/CasY systems, CRISPR/Cmr systems, CRISPR/MAD7 systems, CRISPR/MAD2 systems and/or any combination, variant, or catalytically active fragment thereof.

An “RNA-guided nuclease” is a site-specific nuclease, which requires an RNA molecule, i.e. a guide RNA, to recognize and cleave a specific target site, e.g. in genomic DNA. The RNA-guided nuclease forms a nuclease complex together with the guide RNA and then recognizes and cleaves the target site in a sequence-dependent matter. RNA-guided nucleases can therefore be programmed to target a specific site by the design of the guide RNA sequence.

A “repair template” represents a single-stranded or double-stranded nucleic acid sequence, which can be provided during any genome editing causing a double-strand or single-strand DNA break to assist the targeted repair of said DNA break by providing a RT as template of known sequence assisting homology-directed repair.

A “nucleic acid vector” as used herein refers to a DNA or RNA molecule, which is used to deliver foreign genetic material to a cell, where it can be transcribed and optionally translated. A vector may be a plasmid, a viral vector or an artificial chromosome. Preferably, the vector is a plasmid comprising multiple cloning sites.

A “unique cloning site” is a cloning site that occurs only once in the vector and allows insertion of DNA sequences, e.g. a nucleic acid cassette or components thereof, by use of specific restriction enzymes.

A “flexible insertion site” may be a multiple cloning site, which allows insertion of the components of the nucleic acid cassette according to the invention in an arrangement, which facilitates simultaneous transcription of the components and allows activation of the RNA activation unit.

### DETAILED DESCRIPTION

The present invention provides means and methods for improved genome editing by decreasing the occurrence of non-homology based DNA repair processes during the editing process, which lead to unpredictable and usually undesired outcomes. In particular, non-homologous end joining (NHEJ) and/or microhomology-mediated end joining (MMEJ) pathways are transiently silenced during the editing process, increasing the efficiency and accuracy of the editing. Moreover, the present invention provides means, which are simple to use and allow co-delivery of components within one construct.

According to one aspect, the present invention relates to a nucleic acid cassette, wherein the nucleic acid cassette comprises
(a) at least one RNA interference (RNAi) component;
(b) at least one CRISPR guide RNA component; and
(c) at least one RNA activating unit;
   wherein the nucleic acid cassette is suitable for the simultaneous expression of at least one sequence encoding an RNA interference (RNAi) component and at least one sequence encoding a CRISPR guide RNA component.

Preferably, the at least one RNA interference (RNAi) component encodes a small hairpin RNA (shRNA), which can silence a specific target and the at least one CRISPR guide RNA component encodes a CRISPR RNA (crRNA), which is sufficient for recruiting and guiding its associated RNA-guided site-specific nuclease to its target site and which is processed by its associated RNA-guided nuclease (e.g. Cpf1 or MAD7).

The nucleic acid cassette of the present invention provides a simple construct to be delivered into a cell, from which the silencing component(s) and the CRISPR guide RNA component(s) can be transcribed simultaneously and advantageously under the control of a single promoter. In order for the components to be able to perform their function, i.e. silencing one or more target gene(s) in the cell or, respectively, recruiting and guiding an RNA-guided site-specific nuclease to its designated genomic target site, the transcript needs to be processed to separate the components. Therefore, the nucleic acid cassette comprises an RNA activating unit, which facilitates the processing. The RNA activating unit may comprise a self-cleaving ribozyme system comprising at least one self-cleaving ribozyme, which upon transcription cleaves the transcript at one or more predetermined location(s) to release and thus activate the components (a) and (b). Alternatively, or in addition to the ribozyme system, the RNA activating unit may comprise one or more ribonuclease recognition site(s), which are recognized and processed by an RNA-guided nuclease as in explained in more detail below.

In order to silence non-homologous end joining (NHEJ) and/or microhomology-mediated end joining (MMEJ) pathways, the RNAi component targets one or more component(s), which are involved in the NHEJ and/or MMEJ pathway. The combination of silencing of a NHEJ and/or MMEJ component simultaneously with, or in close temporal relation to a targeted genome editing event can synergistically increase the desired outcome of genome editing in comparison to a conventional genome editing event only aiming at modifying a target site of interest using a site-specific nuclease and optionally a guiding RNA.

In a preferred embodiment of the nucleic acid cassette described above, the RNAi interference component encodes at least one short-hairpin (shRNA) sequence, wherein the at least one shRNA sequence targets at least one non-homologous end joining (NHEJ) pathway component and/or at least one microhomology-mediated end joining (MMEJ) component.

Suitable targets, i.e. components involved in the NHEJ and MMEJ pathway, are described in the prior art and the skilled person is aware how to design an shRNA to target a specific component. Since their role in an NHEJ and MMEJ pathway has been determined, silencing of any one or a combination of several of these components leads to a reduction of NHEJ or MMEJ events during genome editing thus tipping the outcome of a genome editing approach towards a predictable, homology-based outcome.

In one embodiment of the nucleic acid cassette according to any of the embodiments described above, the at least one NHEJ pathway component encodes a sequence selected from the group consisting of Ku70, Ku80, DNA-dependent protein kinase, Ataxia telangiectasia mutated (ATM), ATM—and Rad3—related (ATR), Artemis, XRCC4, DNA ligase IV and XLF, PAXX, or any combination thereof.

In another embodiment of the nucleic acid cassette according to any of the embodiments described above, the at least one MMEJ pathway component encodes a sequence selected from the group consisting of Polymerase theta (pol Θ), an organellar family-A DNA polymerase, DNA ligase III, PARP-1, or any combination thereof.

Advantageously, the nucleic acid cassette according to the invention can be designed to target more than one component of the NHEJ and/or MMEJ pathway, thereby further reducing NHEJ or MMEJ events. In particular, any of the above mentioned NHEJ pathway components and any of the above mentioned MMEJ pathway components can be targeted at the same time by the RNAi component encoded in the nucleic acid cassette.

In one embodiment of the nucleic acid cassette according to any of the embodiments described above, the RNAi interference component encodes more than one short-hairpin (shRNA) targeting more than one NHEJ pathway component(s) and/or MMEJ pathway component(s).

Since KU80 is one of the central components required for NHEJ pathways, it represents a particularly suitable target in order to significantly reduce NHEJ events. As demonstrated in the Examples below, shRNAs targeting KU80 are capable to strongly knock down KU80 (see FIG. 3 and the corresponding description). Another target shown in the Examples is Pol Θ (see FIG. 6 and the corresponding description).

In one embodiment of the nucleic acid cassette according to any of the embodiments described above, the RNAi interference component is encoded by a sequence comprising at least one nucleic acid sequence of any one of SEQ ID NOs: 7 to 12, or SEQ ID NOs: 21 to 26 or a nucleic acid sequence having at least 90%, 91%, 92%, 93%, 94%, 95%, 96%, 97%, 98% or 99% sequence identity to any one of SEQ ID NOs: 7 to 12, or SEQ ID NOs: 21 to 26.

The CRISPR guide RNA component recruits and guides a site-specific CRISPR nuclease in a sequence-dependent manner to a predetermined target site, where the CRISPR nuclease introduces a double-strand break. The CRISPR guide RNA component may comprise a crRNA, which is sufficient for targeting without requiring a tracrRNA, and it may be processed by its associated site-specific nuclease (e.g. Cpf1 or MAD7) without the need for further processing components.

In one embodiment of the nucleic acid cassette according to any of the embodiments described above, the CRISPR guide RNA component comprises a sequence encoding a scaffold region and a targeting region.

The scaffold region represents the recognition and binding site for the RNA-guided site-specific nuclease to form a targetable nuclease complex, which can then perform genome editing. The scaffold region may comprise a nuclease recognition site comprising direct repeats, which are recognized and processed by the site-specific nuclease to provide mature crRNA. For example, Cpf1 and MAD7 can not only cut DNA but they also have ribonuclease activity, which they use to process their pre-crRNA to provide mature crRNA (Safari et al., CRISPR Cpf1 proteins: structure, function and implications for genome editing, Cell & Bioscience (2019), 9:36). In the context of the present invention, the processing by the site-specific nuclease can also be used to release and thus activate the CRISPR guide RNA component from the transcript of the nucleic acid cassette. If processing by the site-specific nuclease is used for activating the crRNA, the RNA-guided site-specific nuclease used for the genome editing needs to be chosen accordingly and the CRISPR guide RNA needs to be designed so that it can be recognized and processed by the RNA-guided site-specific nuclease.

The scaffold region may advantageously be designed for Cpf1 or MAD7 recognition and/or processing. Cpf1 and MAD7 allow specifically simple construct designs because they do not require a tracrRNA for targeting or further components for processing the crRNA since they have ribonuclease activity by which they process their crRNA.

In a further embodiment of the nucleic acid cassette according to any of the embodiments described above, the CRISPR guide RNA component comprises at least one scaffold region encoded by a sequence selected from a nucleic acid sequence of any one of SEQ ID NOs: 29, 30 or 43 or a nucleic acid sequence having at least 90%, 91%, 92%, 93%, 94%, 95%, 96%, 97%, 98% or 99% sequence identity to any one of SEQ ID NOs: 29, 30 or 43.

In one preferred embodiment of the nucleic acid cassette according to any of the embodiments described above, the CRISPR guide RNA component comprises two scaffold regions encoded by sequences selected from a nucleic acid sequence of any one of SEQ ID NOs: 29 and 30 or a nucleic acid sequence having at least 90%, 91%, 92%, 93%, 94%, 95%, 96%, 97%, 98% or 99% sequence identity to any one of SEQ ID NOs: 29 or 30. Preferably, a sequence of SEQ ID NO: 29 (pre-mature MAD7 scaffold) ora sequence having at least 90%, 91%, 92%, 93%, 94%, 95%, 96%, 97%, 98% or 99% sequence identity with SEQ ID NO: 29 (pre-mature MAD7 scaffold) is present at the 5′ end and a sequence of SEQ ID NO: 30 (mature MAD7 scaffold) or a sequence having at least 90%, 91%, 92%, 93%, 94%, 95%, 96%, 97%, 98% or 99% sequence identity with of SEQ ID NO: 30 (mature MAD7 scaffold) is present at the 3′ end of the nucleic acid cassette.

The target region of the CRISPR guide RNA component defines the target site to be modified in the genome editing process. The skilled person is well aware how to design the sequence of the target region to modify a specific genomic target sequence.

In one embodiment of the nucleic acid cassette according to any of the embodiments described above, the CRISPR guide RNA component comprises at least one targeting region encoded by a sequence selected from a nucleic acid sequence of any one of SEQ ID NOs: 13, 14, 27, or 28 or a nucleic acid sequence having at least 90%, 91%, 92%, 93%, 94%, 95%, 96%, 97%, 98% or 99% sequence identity to any one of SEQ ID NOs: 13, 14, 27, or 28.

In order to activate the components of the transcript of the nucleic acid cassette according to the invention, one or more self-cleaving ribozyme(s) may be comprised in the RNA activating unit. Upon transcription, self-cleaving ribozymes fold into a specific structure, which induces cleavage of the phosphodiester backbone at a specific position. By including one or more self-cleaving ribozyme sequences strategically, the transcript is processed according to a predetermined scheme upon transcription, releasing and thus activating components (a) and (b).

In one embodiment of the nucleic acid cassette according to any of the embodiments described above, the at least one RNA activating unit comprises at least one ribozyme system, wherein the at least one ribozyme system comprises at least one sequence encoding a self-cleaving ribozyme.

A self-cleaving ribozyme system may comprise at least one pair of self-cleaving ribozymes, which e.g. flank one or more components (a) and/or (b) in the nucleic acid cassette according to the invention. A self-cleaving ribozyme system may also comprise three or more self-cleaving ribozymes that are arranged in the nucleic acid cassette so that they can activate the component(s) as required.

In one embodiment of the nucleic acid cassette according to any of the embodiments described above, the at least one RNA activating unit comprises at least one ribozyme system comprising at least a pair of sequences encoding two self-cleaving ribozymes, or wherein the ribozyme system comprises at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or more, sequences encoding a self-cleaving ribozyme.

Several suitable ribozyme sequences may be trans-acting or cis-acting as described in the prior art, from which ribozyme sequences a skilled person can choose provided that the ribozyme sequences have the catalytic activity as disclosed herein.

In one embodiment of the nucleic acid cassette according to any of the embodiments described above, the at least one self-cleaving ribozyme of the ribozyme system, or the sequence encoding the same, is independently selected from the group consisting of a hammerhead ribozyme (SEQ ID NOs: 31 and 32), a hairpin ribozyme, a hepatitis-delta virus (HDV) ribozyme (SEQ ID NOs: 41 and 42), a sunflower ribozyme (SEQ ID NOs: 35 to 38), an artichoke ribozyme (SEQ ID NOs: 39 and 40), a rice ribozyme (SEQ ID NOs: 33 and 34), a VS ribozyme, a self-splicing group I intron, or RNase P, a ribozyme sequence of Tobacco ringspot virus (TRSV) satellite RNA or a combination thereof.

The respective ribozymes mentioned above may also be present as a catalytically active fragment of the respective sequences given. Further, the at least one ribozyme sequence may be further modified, for example, by a functional group, by an aptamer, etc.

It is also possible that the RNA activating unit comprises a ribonuclease recognition site, which is recognized and cleaved by the RNA-guided site-specific nuclease used for the genome editing. In this case, the scaffold region of the CRISPR guide RNA component, in particular encoding a pre-crRNA, is recognized and processed by the RNA-guided site-specific nuclease to provide mature crRNA. The processing involves cleavage of the transcript of the nucleic acid cassette thus releasing the crRNA and separating it from other component(s). The scaffold region may comprise direct repeats, which are recognized and processed by Cpf1 (Safari et al. 2019, supra) or MAD7.

In one embodiment of the nucleic acid cassette according to any of the embodiments described above, the at least one RNA activating unit comprises at least one ribonuclease recognition site, wherein the at least one ribonuclease recognition site is recognized and cleaved by at least one RNA-guided site-specific nuclease (SSN) guided by the at least one CRISPR guide RNA component.

Notably, it is also possible that the RNA activating unit comprises both, at least one ribozyme system and at least one ribonuclease recognition site. According to one embodiment of the nucleic acid cassette according to any of the embodiments described above, the RNA activating unit comprises at least one ribozyme system and one or more ribonuclease recognition site(s).

In one embodiment of the nucleic acid cassette according to any of the embodiments described above, the sequence encoding the at least one RNA interference (RNAi) component and the at least one CRISPR guide RNA component is flanked by a sequence encoding a self-cleaving ribozyme at the 5′ end and at the 3′ end and a ribonuclease recognition site is encoded between the at least one RNA interference (RNAi) component and the at least one CRISPR guide RNA component.

In another embodiment of the nucleic acid cassette according to any of the embodiments described above, the sequence encoding the at least one RNA interference (RNAi) component and the at least one CRISPR guide RNA component is flanked by a sequence encoding a ribonuclease recognition site at the 5′ end and at the 3′ end and another ribonuclease recognition site is encoded between the at least one RNA interference (RNAi) component and the at least one CRISPR guide RNA component.

The nucleic acid cassette according to the present invention may also comprise more than one RNAi component and/or more than one CRISPR guide RNA component. In this case, several arrangements are possible. The sequence encoding an RNAi component and encoding a CRISPR guide RNA component may each be flanked by a self-cleaving ribozyme at the 5′ and 3′ end, or a sequence encoding an RNAi component and encoding a CRISPR guide RNA component may be flanked by a self-cleaving ribozyme at the 5′ and 3′ end. In the second case, the CRISPR guide RNA component may be released by processing of the RNA-guided site-specific nuclease.

In one embodiment of the nucleic acid cassette according to any of the embodiments described above, the nucleic acid cassette comprises more than one RNA interference (RNAi) component and/or more than one CRISPR guide RNA component, wherein the sequence encoding an RNAi component and encoding a CRISPR guide RNA component are
(i) each flanked by a sequence encoding a self-cleaving ribozyme at the 5′ and at the 3′ end, or wherein
(ii) a sequence encoding an RNAi component and encoding a CRISPR guide RNA component is flanked by a sequence encoding a self-cleaving ribozyme at the 5′ and at the 3′ end;
   wherein the self-cleaving ribozyme is independently selected from the group consisting of a hammerhead ribozyme (SEQ ID NOs: 31 and 32), a hairpin ribozyme, a hepatitis-delta virus (HDV) ribozyme (SEQ ID NOs: 41 and 42), a sunflower ribozyme (SEQ ID NOs: 35 to 38), an artichoke ribozyme (SEQ ID NOs: 39 and 40), a rice ribozyme (SEQ ID NOs: 33 and 34) a VS ribozyme, a self-splicing group I intron, RNase P, or a ribozyme sequence of Tobacco ringspot virus (TRSV) satellite RNA.

The respective ribozymes mentioned above may also be present as a catalytically active fragment of the respective sequences given.

Preferably, in the second arrangement described above under (ii), the sequence encoding an RNAi component and the sequence encoding a CRISPR guide RNA is separated by a ribonuclease recognition site, which will be processed by the RNA-guided site-specific nuclease used for genome editing to separate the components.

Preferably, the nucleic acid cassette according to the present invention comprises only a single promoter capable of driving the simultaneous expression of the components (a), (b) and (c). Thus, the construct is fairly simple and can easily be employed in a number of settings.

In one embodiment of the nucleic acid cassette according to any of the embodiments described above, the nucleic acid cassette comprises a single promoter driving the expression of the at least one RNA interference (RNAi) component, the at least one CRISPR guide RNA component, and the at least one RNA activating unit.

Suitable promoters are available to the skilled person and may be chosen depending on the setting, in which the nucleic acid cassette according to the invention is used. Furthermore, the nucleic acid cassette may comprise an intron, which may enhance the expression of the nucleic acid cassette according to the invention.

In one embodiment of the nucleic acid cassette according to any of the embodiments described above, the nucleic acid cassette comprises a promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, ora combination thereof.

In another embodiment of the nucleic acid cassette according to any of the embodiments described above, the nucleic acid cassette comprises at least one intron. The intron may be selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

Certain combinations of promoters and introns are particularly preferred as they enhance the expression of the nucleic acid cassette and can thus increase the knockdown effect. According to one embodiment of the nucleic acid cassette according to any of the embodiments described above, the nucleic acid cassette comprises a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and an AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

In addition, the nucleic acid cassette may comprise at least one terminator, which mediates transcriptional termination at the end of the nucleic acid cassette and release of the transcript from the transcriptional complex.

In one embodiment of the nucleic acid cassette according to any of the embodiments described above, the nucleic acid cassette comprises at least one terminator selected from the group consisting of nosT, a double 35S terminator, a ZmEf1 terminator, an AtSac66 terminator, an octopine synthase (ocs) terminator, or a pAG7 terminator, or a combination thereof.

In another aspect, the present invention relates to a vector comprising the nucleic acid cassette according to any of the embodiments described above.

A vector according to the invention is preferably a plasmid, which can be transformed into a cell, in particular a eukaryotic cell, for example an animal or plant cell. The vector allows co-delivery of an RNAi component for silencing NHEJ and/or MMEJ pathways and a CRISPR guide RNA component for site-specific genome editing by an RNA-guided nuclease. The vector therefore facilitates efficient genome editing in a target cell with a predictable homology directed outcome.

In certain embodiments, the nucleic acid cassette may also be introduced into a cell as RNA molecule.

For insertion of the nucleic acid cassette of the present invention into a vector of the present invention, the vector comprises specific restriction sites.

To construct a vector according to the present invention, a standard vector can be modified by inserting a unique cloning site, i.e. by adding further restriction sites. A standard vector may comprise a sequence of SEQ ID NOs: 1 or 15 or a sequence having at least 90%, 91%, 92%, 93%, 94%, 95%, 96%, 97%, 98% or 99% sequence identity to any of SEQ ID NOs: 1 or 15. After insertion of further restriction sites the vector comprises a sequence of SEQ ID NOs: 2 or 16 ora sequence having at least 90%, 91%, 92%, 93%, 94%, 95%, 96%, 97%, 98% or 99% sequence identity to any of SEQ ID NOs: 2 or 16. The resulting vector comprising a sequence of SEQ ID NOs: 2 or 16 ora sequence having at least 90%, 91%, 92%, 93%, 94%, 95%, 96%, 97%, 98% or 99% sequence identity to any of SEQ ID NOs: 2 or 16 provides flexible insertion sites for inserting one or more short-hairpin (shRNA) sequence(s) of an RNA interference (RNAi) component and/or one or more targeting region(s) of a CRISPR guide RNA component as explained in Examples 1 and 2 below.

According to one aspect, the present invention also provides a vector comprising flexible insertion sites for inserting one or more short-hairpin (shRNA) sequence(s) of an RNA interference (RNAi) component and/or one or more targeting region(s) of a CRISPR guide RNA component as explained in Examples 1 and 2 below.

In one embodiment the vector comprises a sequence of SEQ ID NOs: 2 or 16 ora sequence having at least 90%, 91%, 92%, 93%, 94%, 95%, 96%, 97%, 98% or 99% sequence identity to any of SEQ ID NOs: 2 or 16.

In a further aspect, the present invention relates to a method for the targeted modification of at least one genomic target sequence in a cell, wherein the method comprises the following steps:
(a) introducing into the cell at least one nucleic acid cassette as described in any of the embodiments above under conditions allowing the expression of the at least one RNA interference (RNAi) component, the at least one CRISPR guide RNA component, and the at least one RNA activating unit, and thus under conditions allowing activation of the at least one RNA activating unit;
(b) transiently silencing at least one non-homologous end joining (NHEJ) pathway component and/or at least one microhomology-mediated end joining (MMEJ) component through the action of at least one short-hairpin (shRNA) sequence encoded by the at least one RNA interference (RNAi) component;
(c) introducing into the cell at least one site-specific nuclease (SSN), or a sequence encoding the same, wherein the site-specific nuclease has the capability of inducing a single- or double-strand break at a genomic target sequence upon interaction with a sequence encoded by the at least one CRISPR guide RNA component;
(d) optionally: determining the expression level of the at least one NHEJ pathway component and/or at least one MMEJ component, and/or determining the presence of the at least one targeted modification of at least one genomic target sequence in the cell;
(e) obtaining a cell comprising a targeted modification of at least one genomic target sequence.

The method according to the invention allows to perform genome editing while transiently silencing NHEJ and/or MMEJ pathways in order to obtain a more accurate result. The nucleic acid cassette provides a simple construct to be delivered into the target cell, from which the silencing component(s) and the CRISPR guide RNA component(s) can be transcribed simultaneously. The nucleic acid cassette may be delivered into the cell in DNA or RNA form, i.e., in a vector as described above, or as a suitable RNA molecule. The conditions in the cell must be suitable to allow transcription of the components (a), (b) and (c) of the nucleic acid cassette and also allow activation of the RNA activation unit. This may e.g. require the presence of certain metal ion cations to induce the correct folding for self-cleavage or processing by a CRISPR nuclease. Once the RNAi component is active, it temporarily reduces the expression of NHEJ and/or MMEJ pathway components in order to reduce or prevent such repair mechanisms to interfere with the genome editing. An RNA-guided site-specific nuclease is introduced into the cell, simultaneously with, before or after introducing the nucleic acid cassette, depending on the state (RNA/DNA/protein) the RNA-guided site-specific nuclease, or the sequence encoding the same, is introduced into a cell, which RNA-guided site-specific nuclease optionally processes the CRISPR guide RNA component(s), and which is guided by the CRISPR guide RNA component(s) to its designated target site in the genome of the cell. Since components of the non-homologous repair pathway(s) are silenced, the double-strand break introduced by the site-specific nuclease is predominantly repaired by homology-directed processes, e.g. using a repair template.

In one embodiment of the method described above, the method additionally comprises the introduction of at least one single- or double-stranded repair template (RT), or a sequence encoding the same, simultaneously with, or before, or after step (a).

In the presence of a repair template, the double-strand break introduced by the site-specific nuclease is repaired by homologous recombination between the genomic target site and the repair template. Thus, it is possible to introduce a targeted modification, e.g. an insertion of a specific sequence, at the target site.

Preferably, in the method according to any of the embodiments described above, the at least one RNA interference (RNAi) component encodes a small hairpin RNA (shRNA), which can silence a specific target and the at least one CRISPR guide RNA component encodes a CRISPR RNA (crRNA), which is sufficient for recruiting and guiding its associated RNA-guided site-specific nuclease to its target site and which is processed by its associated RNA-guided nuclease (e.g. Cpf1 or MAD7).

In one embodiment of the method according to any of the embodiments described above, the RNAi interference component encodes at least one short-hairpin (shRNA) sequence, wherein the at least one shRNA sequence targets at least one non-homologous end joining (NHEJ) pathway component and/or at least one microhomology-mediated end joining (MMEJ) component.

In another embodiment of the method according to any of the embodiments described above, the at least one NHEJ pathway component encodes a sequence selected from the group consisting of Ku70, Ku80, DNA-dependent protein kinase, Ataxia telangiectasia mutated (ATM), ATM—and Rad3—related (ATR), Artemis, XRCC4, DNA ligase IV and XLF, PAXX, or any combination thereof.

In another embodiment of the method according to any of the embodiments described above, the at least one MMEJ pathway component encodes a sequence selected from the group consisting of Polymerase theta (pol Θ), an organellar family-A DNA polymerase, DNA ligase III, PARP-1, or any combination thereof.

The nucleic acid cassette used in the method according to the invention can be designed to target more than one component of the NHEJ and/or MMEJ pathway, thereby further reducing NHEJ or MMEJ events. In particular, any of the above mentioned NHEJ pathway components and any of the above mentioned MMEJ pathway components can be targeted at the same time by the RNAi component encoded in the nucleic acid cassette.

In one embodiment of the method according to any of the embodiments described above, the RNAi interference component encodes more than one short-hairpin (shRNA) targeting more than one NHEJ pathway component(s) and/or MMEJ pathway component(s).

In another embodiment of the method according to any of the embodiments described above, the RNAi interference component is encoded by a sequence comprising at least one nucleic acid sequence of any one of SEQ ID NOs: 7 to 12, or SEQ ID NOs: 21 to 26 or a nucleic acid sequence having at least 90%, 91%, 92%, 93%, 94%, 95%, 96%, 97%, 98% or 99% sequence identity to any one of SEQ ID NOs: 7 to 12, or SEQ ID NOs: 21 to 26.

In a further embodiment of the method according to any of the embodiments described above, the CRISPR guide RNA component comprises a sequence encoding a scaffold region and a targeting region.

In one embodiment of the method according to any of the embodiments described above, the CRISPR guide RNA component comprises at least one scaffold region encoded by a sequence selected from a nucleic acid sequence of any one of SEQ ID NOs: 29, 30 or 43 or a nucleic acid sequence having at least 90%, 91%, 92%, 93%, 94%, 95%, 96%, 97%, 98% or 99% sequence identity to any one of SEQ ID NOs: 29, 30 or 43.

In one preferred embodiment of the method according to any of the embodiments described above, the CRISPR guide RNA component comprises two scaffold regions encoded by sequences of SEQ ID NOs: 29 and 30 ora nucleic acid sequence having at least 90%, 91%, 92%, 93%, 94%, 95%, 96%, 97%, 98% or 99% sequence identity to any one of SEQ ID NOs: 29 or 30. Preferably, a sequence of SEQ ID NO: 29 (pre-mature MAD7 scaffold) or a sequence having at least 90%, 91%, 92%, 93%, 94%, 95%, 96%, 97%, 98% or 99% sequence identity with SEQ ID NO: 29 (pre-mature MAD7 scaffold) is present at the 5′ end and a sequence of SEQ ID NO: 30 (mature MAD7 scaffold) or a sequence having at least 90%, 91%, 92%, 93%, 94%, 95%, 96%, 97%, 98% or 99% sequence identity with of SEQ ID NO: 30 (mature MAD7 scaffold) is present at the 3′ end of the nucleic acid cassette.

In another embodiment of the method according to any of the embodiments described above, the CRISPR guide RNA component comprises at least one targeting region encoded by a sequence selected from a nucleic acid sequence of any one of SEQ ID NOs: 13, 14, 27, or 28 ora nucleic acid sequence having at least 90%, 91%, 92%, 93%, 94%, 95%, 96%, 97%, 98% or 99% sequence identity to any one of SEQ ID NOs: 13, 14, 27, or 28.

In one embodiment of the method according to any of the embodiments described above, the at least one RNA activating unit comprises at least one ribozyme system, wherein the at least one ribozyme system comprises at least one sequence encoding a self-cleaving ribozyme.

In another embodiment of the method according to any of the embodiments described above, the at least one RNA activating unit comprises at least one ribozyme system comprising at least a pair of sequences encoding two self-cleaving ribozymes, or wherein the ribozyme system comprises at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or more, sequences encoding a self-cleaving ribozyme.

In a further embodiment of the method according to any of the embodiments described above, the at least one self-cleaving ribozyme of the ribozyme system, or the sequence encoding the same, is independently selected from the group consisting of a hammerhead ribozyme (SEQ ID NOs: 31 and 32), a hairpin ribozyme, a hepatitis-delta virus (HDV) ribozyme (SEQ ID NOs: 41 and 42), a sunflower ribozyme (SEQ ID NOs: 35 to 38), an artichoke ribozyme (SEQ ID NOs: 39 and 40), a rice ribozyme (SEQ ID NOs: 33 and 34), a VS ribozyme, a self-splicing group I intron, or RNase P, a ribozyme sequence of Tobacco ringspot virus (TRSV) satellite RNA or a combination thereof.

The respective ribozymes mentioned above may also be present as a catalytically active fragment of the respective sequences given.

In one embodiment of the method according to any of the embodiments described above, the at least one RNA activating unit comprises at least one ribonuclease recognition site, wherein the at least one ribonuclease recognition site is recognized and cleaved by at least one RNA-guided site-specific nuclease (SSN) guided by the at least one CRISPR guide RNA component.

Notably, it is also possible that the RNA activating unit comprises both, at least one ribozyme system and at least one ribonuclease recognition site. According to one embodiment of the nucleic acid cassette according to any of the embodiments described above, the RNA activating unit comprises at least one ribozyme system and one or more ribonuclease recognition site(s).

In one embodiment of the method according to any of the embodiments described above, the sequence encoding the at least one RNA interference (RNAi) component and the at least one CRISPR guide RNA component is flanked by a sequence encoding a self-cleaving ribozyme at the 5′ end and at the 3′ end and a ribonuclease recognition site is encoded between the at least one RNA interference (RNAi) component and the at least one CRISPR guide RNA component.

In another embodiment of the method according to any of the embodiments described above, the sequence encoding the at least one RNA interference (RNAi) component and the at least one CRISPR guide RNA component is flanked by a sequence encoding a ribonuclease recognition site at the 5′ end and at the 3′ end and another ribonuclease recognition site is encoded between the at least one RNA interference (RNAi) component and the at least one CRISPR guide RNA component.

The nucleic acid cassette introduced into the cell in step (a) of the method according to the invention may also comprise more than one RNAi component and/or more than one CRISPR guide RNA component. In this case, several arrangements are possible. The sequence encoding an RNAi component and encoding a CRISPR guide RNA component may each be flanked by a self-cleaving ribozyme at the 5′ and 3′ end, or a sequence encoding an RNAi component and encoding a CRISPR guide RNA component may be flanked by a self-cleaving ribozyme at the 5′ and 3′ end. In the second case, the CRISPR guide RNA component may be released by processing of the RNA-guided site-specific nuclease.

In another embodiment of the method according to any of the embodiments described above, the nucleic acid cassette comprises more than one RNA interference (RNAi) component and/or more than one CRISPR guide RNA component, wherein the sequence encoding an RNAi component and encoding a CRISPR guide RNA component are
(i) each flanked by a sequence encoding a self-cleaving ribozyme at the 5′ and at the 3′ end, or wherein
(ii) a sequence encoding an RNAi component and encoding a CRISPR guide RNA component is flanked by a sequence encoding a self-cleaving ribozyme at the 5′ and at the 3′ end;
   wherein the self-cleaving ribozyme is independently selected from the group consisting of a hammerhead ribozyme (SEQ ID NOs: 31 and 32), a hairpin ribozyme, a hepatitis-delta virus (HDV) ribozyme (SEQ ID NOs: 41 and 42), a sunflower ribozyme (SEQ ID NOs: 35 to 38), an artichoke ribozyme (SEQ ID NOs: 39 and 40), a rice ribozyme (SEQ ID NOs: 33 and 34), a VS ribozyme, a self-splicing group I intron, RNase P, or a ribozyme sequence of Tobacco ringspot virus (TRSV) satellite RNA.

The respective ribozymes mentioned above may also be present as a catalytically active fragment of the respective sequences given.

Preferably, in the second arrangement described above under (ii), the sequence encoding an RNAi component and the sequence encoding a CRISPR guide RNA is separated by a ribonuclease recognition site, which will be processed by the site-specific nuclease added in step (c) to separate the components.

In a further embodiment of the method according to any of the embodiments described above, the nucleic acid cassette comprises a single promoter driving the expression of the at least one RNA interference (RNAi) component, the at least one CRISPR guide RNA component, and the at least one RNA activating unit.

In one embodiment of the method according to any of the embodiments described above, the nucleic acid cassette comprises a promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof.

In another embodiment of the method according to any of the embodiments described above, the nucleic acid cassette comprises at least one intron.

In yet another embodiment of the method described above, the at least one intron is selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

In one embodiment of the method according to any of the embodiments described above, the nucleic acid cassette comprises a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and an AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

In another embodiment of the method according to any of the embodiments described above, the nucleic acid cassette comprises at least one terminator selected from the group consisting of nosT, a double 35S terminator, a ZmEf1 terminator, an AtSac66 terminator, an octopine synthase (ocs) terminator, or a pAG7 terminator, or a combination thereof.

In one embodiment of the method according to any of the embodiments described above, the at least one site-specific nuclease (SSN) is an RNA-guided nuclease.

An RNA-guided nuclease requires an RNA molecule, i.e. a guide RNA, to recognize and cleave a specific target site, e.g. in genomic DNA. The guide RNA may comprise a trans-activating CRISPR RNA (tracrRNA) and a CRISPR RNA (crRNA) or it may be a single guide RNA (sgRNA) consisting of a crRNA and a tracrRNA in one construct. Preferably, the RNA-guided nuclease requires only a crRNA, which is the case for Cpf1 and MAD7. The RNA-guided nuclease forms a nuclease complex together with the guide RNA and then recognizes and cleaves the target site in a sequence-dependent matter. RNA-guided nucleases can be programmed to target a specific site by the design of the guide RNA sequence.

In one embodiment of the method according to any of the embodiments described above, the at least one site-specific nuclease (SSN) is selected from a nuclease from a CRISPR/Cas system, preferably from a CRISPR/Cfp1 system, a CRISPR/MAD7 system, a CRISPR/MAD2 system, a CRISPR/Cas9 system, a CRISPR/CasX system, or a CRISPR/CasY system. The SSN may be introduced into a cell of interest directly as protein, or as DNA/RNA sequence encoding the SSN.

In case processing of the transcript is required for activation of the components of the nucleic acid cassette, the site-specific nuclease (SSN) nuclease has to be selected according to its capability to recognize the ribonuclease recognition site in the RNA activating unit and to process the CRISPR guide RNA component, which is used in the method.

In one embodiment of the method according to any of the embodiments described above, at least one site-specific nuclease (SSN) is selected, which can recognize the at least one ribonuclease recognition site of the RNA activating unit and process the CRISPR guide RNA component to provide a mature crRNA.

Particularly preferred in the method of the present invention is a site-specific nuclease (SSN) selected from a nuclease from a CRISPR/Cfp1 system or a CRISPR/MAD7 system.

Cpf1 and MAD7 nucleases are both classified as Cas12a. They have the advantage that they do not require a tracrRNA but merely need a crRNA for targeting and thus allow a simpler construct design. Moreover, they have ribonuclease activity and can process the pre-crRNA to provide mature crRNA. Therefore, no other effectors need to be added for processing of the crRNA.

In one embodiment of the method according to any of the embodiments described above, the at least one site-specific nuclease (SSN) is selected from the group consisting of a CRISPR/Cfp1 system from Lachnospiraceae bacterium ND2006 (LbCpf1), a CRISPR/Cfp1 system from an Acidaminococcus sp. BV3L6 (AsCpf1), or a CRISPR/MAD7 system from Eubacterium rectale.

The site specific nuclease may be introduced simultaneously with the nucleic acid cassette of the present invention, or it may be introduced before or after the nucleic acid cassette of the present invention is introduced into the cell.

In one embodiment of the method according to any of the embodiments described above, the at least one nucleic acid cassette and/or the at least one site-specific nuclease (SSN), or the sequence encoding the same, are stably or transiently introduced and expressed in the cell.

In another embodiment of the method according to any of the embodiments described above, the targeted modification of the at least one genomic target sequence in a cell is selected from at least one point mutation, at least one insertion, or at least one deletion, or any combination thereof.

The method according to the invention can be used to introduce targeted modification(s) in the genome of different cells. In one embodiment of the method according to any of the embodiments described above, the cell is a eukaryotic cell, preferably a plant cell, or an animal cell.

Particularly preferred in the context of the present invention is the introduction of targeted modification(s) in plants, especially crop plants. The method according to the invention can thus be used to introduce certain desirable traits into a plant. For example, a transgene may be introduced, wherein the transgene or part of the transgene is selected from the group consisting of a gene encoding resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content.

In one embodiment of the method according to any of the embodiments described above, the cell is a plant cell originating from a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In a further aspect, the present invention relates to a cell, preferably a eukaryotic cell selected from a plant cell or an animal cell, obtainable by a method as described in any of the embodiments above.

In yet a further aspect, the present invention relates to an organism, or part of an organism, obtainable by cultivating a cell obtainable by a method as described in any of the embodiments above.

In one embodiment, the cell may be derived from or the organism may be selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In another aspect, the present invention relates to a method of producing a nucleic acid cassette as described in any of the embodiments above.

The nucleic acid cassette according to the invention may be assembled by molecular cloning techniques. For example, once the components (a), (b) and (c) have been designed, and their arrangement in the nucleic acid cassette has been determined, a vector comprising a multiple cloning site can be used to insert the components using specific restriction enzymes.

In one embodiment of the method of producing a nucleic acid cassette as described above, the method comprises a step of inserting at least one unique cloning site into a nucleic acid vector to provide at least one flexible insertion site for at least one short-hairpin (shRNA) sequence of an RNA interference (RNAi) component and/or for at least one targeting region of a CRISPR guide RNA component.

In the method of producing a nucleic acid cassette as described in any of the embodiments above, a standard vector can be modified by inserting a unique cloning site, i.e. by adding restriction sites. A standard vector may comprise a sequence of SEQ ID NOs: 1 or 15 or a sequence having at least 90%, 91%, 92%, 93%, 94%, 95%, 96%, 97%, 98% or 99% sequence identity to any of SEQ ID NOs: 1 or 15. After insertion of further restriction sites the vector comprises a sequence of SEQ ID NOs: 2 or 16 or a sequence having at least 90%, 91%, 92%, 93%, 94%, 95%, 96%, 97%, 98% or 99% sequence identity to any of SEQ ID NOs: 2 or 16. The resulting vector comprising a sequence of SEQ ID NOs: 2 or 16 or a sequence having at least 90%, 91%, 92%, 93%, 94%, 95%, 96%, 97%, 98% or 99% sequence identity to any of SEQ ID NOs: 2 or 16 provides flexible insertion sites for inserting one or more short-hairpin (shRNA) sequence(s) of an RNA interference (RNAi) component and/or one or more targeting region(s) of a CRISPR guide RNA component as explained in Examples 1 and 2 below.

In one embodiment of the method of producing a nucleic acid cassette as described in any of the embodiments above, the method comprises a step of inserting at least one unique cloning site into a nucleic acid vector comprising a sequence of SEQ ID NOs: 1 or 15 or a sequence having at least 90%, 91%, 92%, 93%, 94%, 95%, 96%, 97%, 98% or 99% sequence identity to any of SEQ ID NOs: 1 or 15 to provide at least one flexible insertion site for at least one short-hairpin (shRNA) sequence of an RNA interference (RNAi) component and/or for at least one targeting region of a CRISPR guide RNA component, wherein the vector having the at least one flexible insertion site comprises a sequence of SEQ ID NOs: 2 or 16 or a sequence having at least 90%, 91%, 92%, 93%, 94%, 95%, 96%, 97%, 98% or 99% sequence identity to any of SEQ ID NOs: 2 or 16.

The flexible insertion site allows easy insertion of the least one short-hairpin (shRNA) sequence of an RNA interference (RNAi) component and/or the at least one targeting region of a CRISPR guide RNA component. Furthermore, the RNA activating unit comprising at least one self-cleaving ribozyme and/or at least one ribonuclease recognition site can be included. Thus, the method allows a simple assembly of the nucleic acid cassette of the present invention once the shRNA(s) and the guide RNA(s) have been designed for a specific purpose. The vector can then be transformed into a target cell, where it is transcribed and processed by ribozyme and/or ribonuclease action to activate the components as required.

In yet a further aspect, the present invention also relates to the use of a nucleic acid cassette as described in any of the embodiments above for introducing a targeted modification in at least one genomic target sequence in a cell.

Preferably, the nucleic acid cassette is used in a method for the targeted modification of at least one genomic target sequence in a cell according to any of the embodiments described above.

### EXAMPLES

### Example 1: Utilizing a Ribozyme System and Cpf1 Nuclease RNA Processing to Modulate Maize DNA Repair Pathways Towards SDN-2/3 by RNA Interference

### Construction of Vectors

A vector commonly used here to express LbCpf1 crRNA utilizing Ribozyme processing (pGEP598) was modified to contain BbsI sites in the region between the 5′ Hammerhead ribozyme and LbCpf1 crRNA repeat creating pGEP960. pGEP598 was digested with BamHI and SacI and the vector backbone gel purified. The four oligonucleotides GEP1806 thru GEP1809 (Table 1; SEQ ID NOs: 3-6) were annealed and phosphorylated in a 20 μl reaction in 1× T4 DNA ligase containing 200 picomoles of each oligo and 5U T4 Polynucleotide Kinase by the following thermal cycler program:

1) 37° C. 30 mins
2) 95° C. 5 mins
3) decreasing the temp to 25° C. at a rate of 3° C. per minute.

The annealed, phosphorylated oligos were then cloned into the BamHI and SacI digested pGEP598 by T4 ligation creating pGEP960.

TABLE 1
Oligonucleotides GEP1806 thru GEP1809

| | | |
|---|---|---|
| GEP1806 | gatccaaattaCTGATGAGTCCGTGA | SEQ ID |
| | GGACGAAACGAGTAAGCTCGTCatgt | NO: 3 |
| | cttcatat | |
| GEP1807 | gaagacatTAATTTCTACTAAGTGTA | SEQ ID |
| | GATGAGACGGAGCT | NO: 4 |
| GEP1808 | CGTTTCGTCCTCACGGACTCATCAGt | SEQ ID |
| | aatttg | NO: 5 |
| GEP1809 | CCGTCTCATCTACACTTAGTAGAAAT | SEQ ID |
| | TAatgtcttcatatgaagacatGACG | NO: 6 |
| | AGCTTACT | |

The oligos of Table 2 encoding shRNA (small hairpin RNA) targeted to the ZmKu80 of corn line A188 (GEP1842 thru GEP1847) and crRNA targeted to ZmHMG13 of corn line A188 (crGEP5.Sense and crGEP5.Antisense) were annealed and phosphorylated in sequential pairs by the same methods outlined above.

TABLE 2
Oligonucleotides encoding shRNA targeted to
the ZmKu80 (GEP1842 thru GEP1847) and
crRNA targeted to ZmHMG13 (crGEP5.Sense
and crGEP5.Antisense)

| | | | |
|---|---|---|---|
| siGEP2 | GEP1842 | CGTCGTGCTCCCAGTTA | SEQ ID |
| | | CCGTTTTTCAGATGAAA | NO: 7 |
| | | ACGGTAACTGGGAGCAC | |
| | | TT | |
| | GEP1843 | ATTAAAGTGCTCCCAGT | SEQ ID |
| | | TACCGTTTTCATCTGAA | NO: 8 |
| | | AAACGGTAACTGGGAGC | |
| | | AC | |
| siGEP3 | GEP1844 | CGTCGTCTTGCAGTTTC | SEQ ID |
| | | TGCGATTTCAGATGAAT | NO: 9 |
| | | CGCAGAAACTGCAAGAC | |
| | | TT | |
| | GEP1845 | ATTAAAGTCTTGCAGTT | SEQ ID |
| | | TCTGCGATTCATCTGAA | NO: 10 |
| | | ATCGCAGAAACTGCAAG | |
| | | AC | |
| siGEP4 | GEP1846 | CGTCCGAACATTTACGA | SEQ ID |
| | | GAGATTGATTCAGATGA | NO: 11 |
| | | TCAATCTCTCGTAAATG | |
| | | TTCGTT | |
| | GEP1847 | ATTAAACGAACATTTAC | SEQ ID |
| | | GAGAGATTGATCATCTG | NO: 12 |
| | | AATCAATCTCTCGTAAA | |
| | | TGTTCG | |

| | | |
|---|---|---|
| crGEP5.Sense | TAGATCTCGTCACGATT | SEQ ID |
| | CCCCTCTCCTGG | NO: 13 |
| crGEP5.Antisense | GGCCCCAGGAGAGGGGA | SEQ ID |
| | ATCGTGACGAGA | NO: 14 |

Two enzyme (BbsI and Esp3I) Golden Gate Assembly was used to clone the duplexes as indicated in table 3 into a vector containing the ZmUbi1 promoter+intron driving expression of a Hammerhead Ribozyme+LbCpfl repeat+HDV Ribozyme into a nos terminator as exemplary outlined in the FIG. 2.

TABLE 3
Duplexes of shRNA and crRNA as integrated into vectors

| | shRNA | crRNA |
|---|---|---|
| pGEP986 | siGEP2 | crGEP5 |
| pGEP987 | siGEP3 | crGEP5 |
| pGEP988 | siGEP4 | crGEP5 |

### Transfection of Protoplasts

Each of the plasmids expressing siGEP2 thru siGEP4 (pGEP986 thru pGEP988 respectively) and crGEP5 were inserted into Maize protoplasts by PEG mediated transformation along with a 2^{nd} plasmid expressing a codon optimized LbCpf1 and mNeonGreen (pGEP863) (5 μg PGEP986 thru pGEP988+10 μg pGEP863). A control transfection was also performed with a plasmid only encoding the LbCpf1 guide+repeat (pGEP610) and pGEP863.

Transformation Efficiency was calculated by Flow Cytometry to detect mNeonGreen.

### Determination of Knockdown

RNA was extracted from the protoplasts after 24 hours. Reverse Transcription PCR was used to generate cDNA. cDNA samples were used as template in qPCR to detect KU80 expression levels by delta delta CT analysis with primers for TUA4 as the endogenous control.

All three siRNA expressing plasmids lowered expression levels of KU80. FIG. 3 shows knockdown % of Ku80 RNA caused by protoplast transformation of the plasmids (pGEP986 thru pGEP988) expressing the shRNA encoding sequences (siGEP2 thru siGEP4.) Indel frequency of the LbCpf1 was not significantly negatively affected by the presence of the siRNA encoding sequence between the 5′ Ribozyme and the LbCpf1 repeat. Table 4 shows results from ddPCR analysis for SDN-1 editing at the crGEP5 target site in three replicates of the transformations. Interestingly is the observation that editing ration is increased. This cannot be explained solely by the slight increase in transformation efficiency (TE), so that the normalized editing ratio is around 10% on average higher in Ku70 knockdown lines compared to control.

TABLE 4
SDN-1 editing at the crGEP5 target site in three replicates
of the transformations. Editing Ratio % represents
raw data; Editing Ratio % normalized for TE is normalized
to Transformation Efficiency (TE)

| | Editing | Normalized Editing | |
|---|---|---|---|
| Sample ID | Ratio [%] | Ratio [%] | TE |
| pGEP863 + pGEP610-1 | 20.3 | 56.4 | 0.36 |
| pGEP863 + pGEP610-2 | 25.8 | 68.4 | 0.38 |
| pGEP863 + pGEP610-3 | 23.7 | 57.0 | 0.42 |
| Mean control | 23.3 | 60.6 | 0.39 |
| pGEP863 + pGEP986-1 | 25.8 | 58.2 | 0.44 |
| pGEP863 + pGEP986-2 | 28.6 | 71.3 | 0.40 |
| pGEP863 + pGEP986-3 | 32.1 | 78.7 | 0.41 |
| pGEP863 + pGEP987-1 | 34.3 | 77.4 | 0.44 |
| pGEP863 + pGEP987-2 | 29.2 | 70.4 | 0.42 |
| pGEP863 + pGEP987-3 | 30.8 | 75.1 | 0.41 |
| pGEP863 + pGEP988-1 | 36.2 | 74.5 | 0.49 |
| pGEP863 + pGEP988-2 | 32.7 | 68.4 | 0.48 |
| pGEP863 + pGEP988-3 | 24.6 | 57.1 | 0.43 |
| Mean KU70 knockdown | 30.5 | 70.1 | 0.44 |

### Example 2: Adaption of Dual-Expression of shRNA and crRNA from a Single PoIII Promoter Utilizing Ribozymes to Mad7 Nuclease

Creating a Mad7 Guide Expression Construct with shRNA Expression

A vector used to express Mad7 guide RNA utilizing Ribozyme processing (pGEP832) was modified to contain Esp3I sites in the region between the 5′ Hammerhead ribozyme and Mad7 guide RNA repeat creating pGEP1062. Same methods used as when creating pGEP960 (oligos GEP1901 thru GEP1904) in Example 1.

TABLE 5
Oligonucleotides GEP1901 thru GEP1904

| | | |
|---|---|---|
| GEP1901 | gatccaaattaCTGATGAGTCCGTGAGG | SEQ ID |
| | ACGAAACGAGTAAGCTCGTCagagacgA | NO: 17 |
| | Aata | |
| GEP1902 | tcgtctctGTCAAAAGACCTTTTTAATT | SEQ ID |
| | TCTACTCTTGTAGATGAGACGGAGCT | NO: 18 |
| GEP1903 | ctGACGAGCTTACTCGTTTCGTCCTCAC | SEQ ID |
| | GGACTCATCAGtaatttg | NO: 19 |
| GEP1904 | CCGTCTCATCTACAAGAGTAGAAATTAA | SEQ ID |
| | AAAGGTCTTTTGACagagacgatatTTc | NO: 20 |
| | gtct | |

Construction of Mad7 Guide and shRNA Expressing Plasmids

The following oligos encoding shRNA targeted to the A188 ZmPolTheta and Mad7 crRNA targeted to A188 ZmHMG13 (m7GEP1.Sense and m7GEP1.Antisense or m7GEP22.Sense and m7GEP22.Antisense) were annealed and phosphorylated in sequential pairs by the same methods outlined with the Ku80 experiment.

TABLE 6
Oligonucleotides encoding shRNA
targeted to the PolTheta (GEP1890 thru GEP1892)
and crRNA targeted to ZmHMG13
(crGEP5.Sense and crGEP5.Antisense)

| | | | |
|---|---|---|---|
| siGEP6 | GEP1890 | CGTCGAACTCGCTGATG | SEQ ID |
| | | GGATATCTTTCAGATGA | NO: 21 |
| | | AGATATCCCATCAGCGA | |
| | | GTTCTT | |
| | GEP1911 | TGACAAGAACTCGCTGA | SEQ ID |
| | | TGGGATATCTTCATCTG | NO: 22 |
| | | AAAGATATCCCATCAGC | |
| | | GAGTTC | |
| siGEP7 | GEP1892 | CGTCGATATCAAGTTGG | SEQ ID |
| | | CTTCCTCCTTCAGATGA | NO: 23 |
| | | GGAGGAAGCCAACTTGA | |
| | | TATCTT | |
| | GEP1912 | TGACAAGATATCAAGTT | SEQ ID |
| | | GGCTTCCTCCTCATCTG | NO: 24 |
| | | AAGGAGGAAGCCAACTT | |
| | | GATATC | |
| siGEP8 | GEP1894 | CGTCCTTGGTGTAATTG | SEQ ID |
| | | TCATAGATTTCAGATGA | NO: 25 |
| | | ATCTATGACAATTACAC | |
| | | CAAGTT | |
| | GEP1913 | TGACAACTTGGTGTAAT | SEQ ID |
| | | TGTCATAGATTCATCTG | NO: 26 |
| | | AAATCTATGACAATTAC | |
| | | ACCAAG | |

| | | |
|---|---|---|
| m7GEP1.Sense | tagaTCTCGTCACGATT | SEQ ID |
| | CCCCTCTCC | NO: 27 |
| m7GEP1.Antisense | ggccGGAGAGGGGAATC | SEQ ID |
| | GTGACGAGA | NO: 28 |

Esp3I Golden Gate Assembly was used to clone the duplexes as indicated in table 7 into a vector containing the ZmUbi1 promoter+intron driving expression of a Hammerhead Ribozyme+Mad7 guide repeat+HDV Ribozyme into a nos terminator (pGEP1072) as exemplary outlined in FIG. 5.

TABLE 7
Duplexes of shRNA and M7 guide as integrated into vectors

| | shRNA | M7 guide |
|---|---|---|
| pGEP1072 | siGEP6 | m7GEP1 |
| pGEP1073 | siGEP7 | m7GEP1 |
| pGEP1074 | siGEP8 | m7GEP1 |

### Transfection of Protoplasts

Each of the plasmids expressing the shRNA and Mad7 guides were inserted into Maize protoplasts by PEG mediated transformation along with a 2^{nd} plasmid expressing a codon optimized Mad7 and mNeonGreen (pGEP837) (5 μg pGEP1072 thru pGEP1074+10 μg pGEP837).

A control transfection was also performed with a plasmid only encoding the Mad7 guide+repeat (pGEP842 for pGEP1072 thru pGEP1074) and pGEP837.

Transformation Efficiency was calculated by Flow Cytometry to detect mNeonGreen.

### Determination of Knockdown

RNA was extracted from the protoplasts after 24 hours. Reverse Transcription PCR was used to generate cDNA. cDNA samples were used as template in qPCR to detect PolTheta expression levels by delta delta CT analysis with primers for TUA4 as the endogenous control.

All three shRNA expressing plasmids lowered expression levels of PolTheta. FIG. 6 shows knockdown of PolTheta RNA caused by protoplast transformation of the plasmids (pGEP1072 thru pGEP1074) expressing the shRNA encoding sequences (siGEP6 thru siGEP8).

Indel frequency of the Mad7 was not significantly negatively affected by the presence of the siRNA encoding sequence between the 5′ Ribozyme and Mad7 scaffolding/repeat compared to those obtained thru the vectors lacking them.

### Example 3: Determination of Effects of Knocking Down Repair Pathway mRNA on SDN-213

Various repair templates utilizing Homologous Recombination (HR,) Single Strand Annealing (SSA,) or Microhomology Mediated End-Joining (MMEJ) have been co-bombarded into maize embryos with the plasmids found to have the highest levels of Ku80 and/or PolTheta knockdown effect and the Cpf1 or Mad7 nuclease expression construct, respectively, pGEP863 or pGEP837. As controls, the base guide expression plasmids only containing the Cpf1 guide+repeat or the Mad7 guide+repeat, respectively, have been co-bombarded with pGEP837 and repair templates.

Homologous recombination repair templates for SDN-3 comprises dsDNA with 300-1500 bp homology arms and an in-frame coding sequence (CDS) to express a reporter or selectable marker. These are tested in combination with plasmids either targeting PolTheta, Ku80, or a combination of both.

Single Stranded Annealing repair templates for SDN-2 comprises Single Strand DNA with 30-150 bp homology arms and sequence to introduce various detectable mutations (SNPs, small insertions, small deletions) at the guide RNA target site. These are tested in combination with plasmids either targeting PolTheta, Ku80, or a combination of both.

Microhomology Mediated End-Joining (MMEJ) repair templates for SDN-2 or SDN-3 comprises ssDNA or dsDNA with 5-25 bp homology arms and sequence to introduce either various detectable mutations (SNPs, small insertions, small deletions) at the guide RNA target site or an in-frame coding sequence (CDS) to express a reporter or selectable marker. These are tested in combination with plasmids targeting Ku80 (MMEJ utilizes PolTheta).

### Results

It can be expected that knocking down either, or, both genes will result in a 1.3 to 2 fold increase in SDN-2/3.

### Example 4: Further Improvements

### Optimization of Promoters, Introns, or Terminators

After identifying which shRNA provide the most knockdown of their targets, the plasmid expression system is modified with different promoter and intron, and/or terminator sequences for testing if the knockdown effect can be increased.

Promoter+introns to test: current: ZmUbi1+intron (520 bp intron), ZmUbi1+FL intron (1005 bp), BdUbi10+intron, ZmEf1+intron, 2×355+AdH1 intron, or 2×35S+ZmUbi1 intron

Terminators to test: nosT, 2×355, or ZmEf1

1. A nucleic acid cassette, wherein the nucleic acid cassette comprises
(a) at least one RNA interference (RNAi) component;
(b) at least one CRISPR guide RNA component; and
(c) at least one RNA activating unit;
wherein the nucleic acid cassette is suitable for the simultaneous expression of at least one sequence encoding an RNA interference (RNAi) component and at least one sequence encoding a CRISPR guide RNA component.

2. The nucleic acid cassette of claim 1, wherein the RNAi interference component encodes at least one short-hairpin (shRNA) sequence, wherein the at least one shRNA sequence targets at least one non-homologous end joining (NHEJ) pathway component and/or at least one microhomology-mediated end joining (MMEJ) component.

3. The nucleic acid cassette of claim 2, wherein the at least one NHEJ pathway component encodes a sequence selected from the group consisting of Ku70, Ku80, DNA-dependent protein kinase, Ataxia telangiectasia mutated (ATM), ATM—and Rad3—related (ATR), Artemis, XRCC4, DNA ligase IV and XLF, PAXX, or any combination thereof.

4. The nucleic acid cassette of claim 2, wherein the at least one MMEJ pathway component encodes a sequence selected from the group consisting of Polymerase theta (pol Θ), an organellar family-A DNA polymerase, DNA ligase III, PARP-1, or any combination thereof.

5. The nucleic acid cassette of claim 1, wherein the RNAi interference component encodes more than one short-hairpin (shRNA) targeting more than one NHEJ pathway component(s) and/or MMEJ pathway component(s).

6. The nucleic acid cassette of claim 1, wherein the RNAi interference component is encoded by a sequence comprising at least one nucleic acid sequence of any one of SEQ ID NOs: 7 to 12, or SEQ ID NOs: 21 to 26 or a nucleic acid sequence having at least 90%, 91%, 92%, 93%, 94%, 95%, 96%, 97%, 98% or 99% sequence identity to any one of SEQ ID NOs: 7 to 12, or SEQ ID NOs: 21 to 26.

7. The nucleic acid cassette of claim 1, wherein the CRISPR guide RNA component comprises a sequence encoding a scaffold region and a targeting region.

8. The nucleic acid cassette of claim 1, wherein the CRISPR guide RNA component comprises at least one scaffold region encoded by a sequence selected from a nucleic acid sequence of any one of SEQ ID NOs: 29, 30 or 43 or a nucleic acid sequence having at least 90%, 91%, 92%, 93%, 94%, 95%, 96%, 97%, 98% or 99% sequence identity to any one of SEQ ID NOs: 29, 30 or 43.

9. The nucleic acid cassette of claim 1, wherein the CRISPR guide RNA component comprises at least one targeting region encoded by a sequence selected from a nucleic acid sequence of any one of SEQ ID NOs: 13, 14, 27, or 28 or a nucleic acid sequence having at least 90%, 91%, 92%, 93%, 94%, 95%, 96%, 97%, 98% or 99% sequence identity to any one of SEQ ID NOs: 13, 14, 27, or 28.

10. The nucleic acid cassette of claim 1, wherein the at least one RNA activating unit comprises at least one ribozyme system, wherein the at least one ribozyme system comprises at least one sequence encoding a self-cleaving ribozyme.

11. The nucleic acid cassette of claim 1, wherein the at least one RNA activating unit comprises at least one ribozyme system comprising at least a pair of sequences encoding two self-cleaving ribozymes, or wherein the ribozyme system comprises at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or more, sequences encoding a self-cleaving ribozyme.

12. The nucleic acid cassette of claim 10, wherein the at least one self-cleaving ribozyme of the ribozyme system, or the sequence encoding the same, is independently selected from the group consisting of a hammerhead ribozyme (SEQ ID NOs: 31 and 32), a hairpin ribozyme, a hepatitis-delta virus (HDV) ribozyme (SEQ ID NOs: 41 and 42), a sunflower ribozyme (SEQ ID NOs: 35 to 38), an artichoke ribozyme (SEQ ID NOs: 39 and 40), a rice ribozyme (SEQ ID NOs: 33 and 34), a VS ribozyme, a self-splicing group I intron, or RNase P, a ribozyme sequence of Tobacco ringspot virus (TRSV) satellite RNA or a combination thereof.

13. The nucleic acid cassette of claim 1, wherein the at least one RNA activating unit comprises at least one ribonuclease recognition site, wherein the at least one ribonuclease recognition site is recognized and cleaved by at least one RNA-guided site-specific nuclease (SSN) guided by the at least one CRISPR guide RNA component.

14. The nucleic acid cassette of claim 1, wherein the nucleic acid cassette comprises more than one RNA interference (RNAi) component and/or more than one CRISPR guide RNA component, wherein the sequence encoding an RNAi component and encoding a CRISPR guide RNA component are
(i) each flanked by a sequence encoding a self-cleaving ribozyme at the 5′ and at the 3′ end, or wherein
(ii) a sequence encoding an RNAi component and encoding a CRISPR guide RNA component is flanked by a sequence encoding a self-cleaving ribozyme at the 5′ and at the 3′ end;
wherein the self-cleaving ribozyme is independently selected from the group consisting of a hammerhead ribozyme (SEQ ID NOs: 31 and 32), a hairpin ribozyme, a hepatitis-delta virus (HDV) ribozyme (SEQ ID NOs: 41 and 42), a sunflower ribozyme (SEQ ID NOs: 35 to 38), an artichoke ribozyme (SEQ ID NOs: 39 and 40), a rice ribozyme (SEQ ID NOs: 33 and 34), a VS ribozyme, a self-splicing group I intron, RNase P, or a ribozyme sequence of Tobacco ringspot virus (TRSV) satellite RNA.

15. The nucleic acid cassette of claim 1, wherein the nucleic acid cassette comprises a single promoter driving the expression of the at least one RNA interference (RNAi) component, the at least one CRISPR guide RNA component, and the at least one RNA activating unit.

16. The nucleic acid cassette of claim 1, wherein the nucleic acid cassette comprises a promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof.

17. The nucleic acid cassette of claim 1, wherein the nucleic acid cassette comprises at least one intron.

18. The nucleic acid cassette of claim 17, wherein the at least one intron is selected from the group consisting of a ZmUbi1 intron, an FL intron, a BdUbi10 intron, a ZmEf1 intron, an AdH1 intron, a BdEF1 intron, a MeEF1 intron, an EsEF1 intron, and a HSP70 intron.

19. The nucleic acid cassette of claim 1, wherein the nucleic acid cassette comprises a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and an AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

20. The nucleic acid cassette of claim 1, wherein the nucleic acid cassette comprises at least one terminator selected from the group consisting of nosT, a double 35S terminator, a ZmEf1 terminator, an AtSac66 terminator, an octopine synthase (ocs) terminator, or a pAG7 terminator, or a combination thereof.

21-36. (canceled)
